(19) United States
(12) Patent Application Publication
(10) Pub. No.: US 2015/0297573 A1
(43) Pub. Date: Oct. 22, 2015

(54) TPL2 KINASE INHIBITORS FOR PREVENTING OR TREATING DIABETES AND FOR PROMOTING Beta-CELL SURVIVAL

(71) Applicants: INSERM (Institut National De La Santé Et De La Recherche Médicale), Paris (FR); Université De Montpellier, Montpellier (FR)

(72) Inventors: Stéphane Dalle, Montpellier Cedex (FR); Jean-Francois Tanti, Nice Cedex 3 (FR); Anne Wojtusciszyn, Montpellier Cedex 5 (FR); Elodie Varin, Montpellier Cedex (FR)

(73) Assignees: INSERM (Institut National De La Santé Et De La Recherche Médicale), Paris (FR); Université De Montpellier, Montpellier (FR)

(21) Appl. No.: 14/437,909

(22) PCT Filed: Oct. 24, 2013
(86) PCT No.: PCT/EP2013/072314

§ 371 Date: Apr. 23, 2015

Related U.S. Application Data
(60) Provisional application No. 61/717,828, filed on Oct. 24, 2012.

(30) Foreign Application Priority Data
Oct. 24, 2012 (EP) 12306321.6

(51) Int. Cl. A61K 31/4375; A61K 45/06

(52) U.S. Cl. CPC A61K 31/4375; A61K 45/06

(57) ABSTRACT

The present invention relates to the use of a Tpl2 kinase inhibitor for preventing treating diabetes and promoting β-cell survival and function in a number of applications.

### FIELD OF THE INVENTION

The invention relates to the use of Tpl2 kinase inhibitors for promoting β-cell survival and function. This opens the field of a new treatment for preventing or treating diabetes.

### BACKGROUND OF THE INVENTION

Diabetes is characterized by elevated levels of plasma glucose (hyperglycemia) in the fasting state or after administration of glucose during an oral glucose tolerance test. In type 1 diabetes mellitus (TD1M) or insulin-dependent diabetes mellitus (IDDM), patients produce little or no insulin, the hormone which regulates glucose utilization. T1DM is an autoimmune disease leading to the destruction of β-cells, which are within the pancreatic islets the only insulin-secreting cells in the organism. β-cell attacks are mediated by pro-inflammatory cytokines after auto-immunity activation. In the last decade, pancreatic islet transplantation has emerged as a promising alternative therapy for T1DM. This technique needs isolation of islets from deceased donor pancreas and transplantation of them into patient liver. Successful transplantation can improve glycemic control, relieve the patient from insulin dependence and improve quality of life. However, clinical outcome is not always due to significant loss of islet mass during or after transplantation. The loss of islets is caused by several reasons including instant blood-mediated inflammatory reaction (IBMIR) and adaptive immune response. There is incremental evidence that cytokines play a crucial role in both processes. Cytokines themselves can directly trigger islet cell death. Indeed, cytokines such as IL-1β (Interleukin-1β), TNF-α (Tumor Necrosis Factor-α) and IFN-γ (Interferon-γ) have important pro-inflammatory and pro-apoptotic roles in T1DM and islet transplantation. Although immunosuppressants are administered after transplantation of islets, they do not target cytokine-mediated damages to islets. Anti-TNF is nowadays widely used in islet transplantation for preventing IBMIR: although it improved islet-recipients outcomes, it can not prevent totally the inflammatory phenomenon. Targeting the other cytokines that mediate inflammation in this context would be of great importance.

Moreover, type 2 diabetes mellitus (T2DM) or Non-Insulin-Dependent Diabetes Mellitus (NIDDM) is a serious health problem that by 2030 is projected to affect more than 350 million individuals world-wide, of which around 60 millions will be in Europe. Because of the associated morbidity and mortality, diabetes is one of the top burdens on health and social care systems, and will increase as the population ages. Currently, no available treatment can stop T2DM progression. Current treatments are focused on lowering blood glycemia or reducing insulin-resistance, a therapeutic strategy that is insufficient in preventing subsequent disease progression and downstream diabetic complications. Indeed, international health organizations are strongly recommending the development of treatments acting on the pathogenesis of the disease rather than just on its symptoms. Therefore, innovative therapeutic treatments, beyond today's generation of anti-glycemics, are needed to treat T2DM patient and prevent onset and progression of this disease.

It should be recalled that the pathophysiology of T2DM is characterized by peripheral chronic insulin resistance and a progressive decline of β-cell function and mass (Kudva et al., 1997; DeFronzo 1988). Obesity is a major risk factor for the development of T2DM (Burke et al., 1999; CDC 1997) and is thought to confer increased risk for T2DM through the obesity-associated insulin resistance (Ludvik et al., 1995). However, most people who are obese (and relatively insulin resistant) do not develop diabetes but compensate by increasing insulin secretion from β-cells (Polonsky 2000). Hence, it is now well accepted that the insulin resistance of T2DM, although important for its pathophysiology, is not sufficient to establish the disease unless major deficiency of β-cell function co-exists (Butler et al., 2003; Leibowitz and al., 2009). Consequently, pharmacological strategies aiming at improving increased β-cell function and survival in people with a defined high risk for developing T2DM are essential to slow the progression or even to prevent T2DM.

It should be further noted that although obesity (condition whereby an otherwise healthy subject has a Body Mass Index (BMI) greater than or equal to 30 kg/m²) is a key component of T2DM, particularly in the Western world, many patients are not overweight by traditional criteria. Individuals with T2DM can thus present variable clinical characteristics. Thus there are many lean diabetes patients (people whose BMI is less than 25 kg/m² or even less than 20 kg/m²) and many overweight people without diabetes. The clinical presentation and profile of associated complications is different in lean patients of T2DM, as compared to obese. Thus lean T2DM can be considered is a distinct clinical entity. Lean patients are more likely to be older at diagnosis and may have a tendency towards certain pathophysiological characteristics, notably less insulin resistance and poorer insulin secretory capacity. Several studies suggest poor β-cell function in such lean patients.

Recently, it has become clear that chronic inflammation is a hallmark of T2DM, affecting both the pancreatic β-cell function and mass. As previously mentioned, a patient may thus be become diabetic due to the inability to properly compensate for insulin resistance. In humans, beta cells within the pancreatic islets initially compensate for insulin resistance by increasing insulin output. The onset of T2DM due to insufficient increase (or actual decline) in beta cell mass is therefore due to increased beta cell apoptosis relative to non-diabetic insulin resistant individuals.

Indeed, chronic activation of the innate immune system was found to be associated with a reduction in β-cell function and mass. Pancreatic islets from T2DM patients were found to display elevated levels of pro-inflammatory cytokines such as IL-1β and TNF-α, diverse chemokines, and to be infiltrated with macrophages (Donath and Shoelson, 2011; Dinarello et al, 2010). Long term exposure to high concentrations of IL-1β exerts detrimental effects on β-cell and human islets. Exposure of human islets to metabolic stresses such as elevated glucose (glucotoxicity) and palmitate (lipotoxicity) concentrations increase levels of IL-1β and chemokines. The inflammatory cytokines produced into the islets by macrophages and/or β-cells may both contribute to β-cell death and insulin secretory failure (Donath and Shoelson, 2011; Dinarello et al, 2010; Maedler et al, 2002). Based on these observations, immune-modulatory strategies for the treatment of T2DM have emerged (Böni-Schnetzler et al, 2012; Larsen et al, 2009; Larsen et al, 2007). Very mild reduced hyperglycemia and improved β-cell function were observed in type 2 diabetic patients treated with IL-1β receptor antagonist (IL-1RA) (Larsen et al, 2007) but no real clinical impact was observed. This first gave the proof of concept for the use of immune-modulatory strategies in T2DM, but targeting other cytokines would be probably more efficient.

In these different contexts (T2DM, T1DM, Islet transplantation), protein kinases that specifically control the inflammatory response induced not only by IL-1β but also by other cytokines (TNF-α and IFN-γ) may be interesting targets for therapeutic intervention against β-cell failure. Activation of extracellular signal-regulated kinases (ERK)-1/2 (p44/42 mitogen-activated protein (MAP) kinases) has been reported to play a role in the detrimental effects of IL-1β on β-cells (Maedler et al, 2004). However, it must be noted that depending on the nature of the stimuli, the ERK1/2 pathway is involved in a broad range of biological processes within the 13 cells (Maedler et al, 2004; Costes et al, 2006).

As example, ERK1/2 plays a key role in glucose-mediated β-cell survival (Costes et al, 2006). Together, identification of proteins which regulate ERK1/2 activity specifically in response to cytokines (IL-1β, TNF-α, IFN-γ) not only may provide important new insights into the molecular mechanisms that promote β-cell dysfunction, but also may propose these proteins as therapeutic targets to alleviate β-cell failure in T2DM.

Accordingly, identifying new targets that specifically control the inflammatory response induced pro-inflammatory cytokines IL-1β, TNF-α and IFN-γ may be interesting for an optimal prevention or treatment against diabetes (e.g. T1DM and T2DM) as well as for efficient and safe islet cell transplantation.

Amongst the huge number of potential targetable kinases, Tpl2 kinase was disclosed many years ago as a kinase involved in inflammation via the modulation of NFkB activity since it was shown that Tpl2 kinase is responsible for the degradation of p105 and resultant release of Rel subunits. Accordingly, a rationale for treating autoimmune diseases in which NFkB may be involved such as multiple sclerosis (MS), inflammatory bowel disease (IBS), IDDM (T1DM), psoriasis and rheumatoid arthritis, amongst many others was speculated upon in the US publication No US 2003/0319427 although no relevant results or specific technical support in relation to T1DM were disclosed.

More recently, the role of Tpl2 kinase in mediating obesity-associated insulin-resistance was investigated by using Tpl2 knockout (KO) mice (Perfield et al. 2011) and it was suggested that Tpl2 kinase is a promising therapeutic target for improving the metabolic state associated with obesity and more particularly for restoring insulin sensitivity. However, such preliminary results were contested by another team using the same Tpl2 KO mice since the authors failed to find any significant role in regulating obesity-associated metabolic disorders (Lancaster et al., 2012) and, even more, they concluded that Tpl2 deletion exacerbates the effects of the High Fat (HF) diet (i.e. impaired insulin tolerance, compared to wild type mice). They also observed no HF diet-induced increases in ERK1/2 phosphorylation arguing against a prominent role for Tpl2 kinase in mediating ERK activation in response to diet in vivo. More interestingly, they disclosed that general islet morphology was similar in wt and Tpl2^{-/-} mice.

### SUMMARY OF THE INVENTION

As described in the Examples below, the present inventors have demonstrated that the MAP3 kinase Tpl2 specifically mediates signaling pathways induced by inflammatory cytokines in β-cells, and plays an important role in triggering β-cell dysfunction and destruction, and that Tpl2 kinase inhibitors protect pancreatic β-cells from apoptosis. Accordingly, Tpl2 kinase inhibitors are useful for preventing and treating diabetes and promoting β-cell survival in a number of applications. More specifically the inventors have shown that, unexpectedly, the Tpl2 kinase is expressed in β-cells, mouse and human pancreatic islets, and is specifically involved in ERK1/2 activation by IL-1β alone or a cytokine mixture (IL-1β+TNFα+IFNγ) and have demonstrated that pharmacological inhibition of Tpl2 kinase prevents ERK1/2 activation and the detrimental effects of chronic exposure of IL-1β alone or of a cytokine mixture on β-cells and human pancreatic islets. Importantly, neither glucose-induced ERK1/2 nor p90RSK phosphorylations, described to play a key role in glucose-mediated β-cell survival (Costes et al, 2006), were modified neither by Tpl2 inhibitor treatment.

Prior to the present disclosure, Tpl2 kinase had not been shown to be expressed in β-cells and its role in mediating signaling pathways such as ERK1/2 pathway in response to said three major pro-inflammatory cytokines involved in β-cell dysfunction and apoptosis leading to T2DM was unknown.

These novel findings support novel pharmaceutical interventions for Tpl2 kinase inhibitors e.g. to promote β-cell survival and function, for example by inhibiting β-cell apoptosis. Additionally the invention has utility in increasing the efficiency of islet cell transplantation by promoting graft survival and not obtained by the current immunosuppressive treatments.

Thus a non-limiting list of these and other aspects of the invention is as follows:

In a first aspect, the present invention relates to a Tpl2 (Tumor Progression Locus-2) kinase inhibitor for use in the prevention or treatment of diabetes in a patient in need thereof.

In a second aspect, the present invention relates to an inhibitor of the Tpl2 kinase gene expression for use in the prevention or treatment of diabetes in a patient in need thereof.

In a third aspect, the present invention relates to a Tpl2 kinase inhibitor or an inhibitor of the Tpl2 kinase gene expression for use in improving survival or function of pancreatic β-cells in a patient in need thereof.

In a fourth aspect, the present invention relates to a pharmaceutical composition or a kit-of-part comprising a Tpl2 kinase inhibitor or an inhibitor of the Tpl2 kinase gene expression and an anti-diabetic drug.

In a fifth aspect, the present invention relates to a Tpl2 kinase inhibitor or an inhibitor of the Tpl2 kinase gene expression for use in enhancing the clinical efficacy of an anti-diabetic drug.

In a sixth aspect, the present invention relates Tpl2 kinase inhibitor or an inhibitor of the Tpl2 kinase gene expression for use in enhancing the anti-inflammatory action and/or the preservation of pancreatic β-cell viability and/or function of an anti-diabetic drug.

In a seventh aspect, the present invention relates to a culture medium suitable for the culture of mammalian pancreatic β-cells comprising a Tpl2 kinase inhibitor or an inhibitor of the Tpl2 kinase gene expression.

In a eighth aspect, the present invention relates to a method for improving survival and/or function of a population of pancreatic β-cells in vitro or ex vivo, said method comprising a step of contacting said population with a culture medium comprising an effective amount of a Tpl2 kinase inhibitor or an inhibitor of the Tpl2 kinase gene expression.

In a ninth aspect, the present invention relates to a method for improving survival and/or function of a pancreatic β-cell transplant, said method comprising a step of administering an effective amount of Tpl2 kinase inhibitor or an inhibitor of the Tpl2 kinase gene expression to a patient with a pancreatic β-cell transplant.

In still another aspect, the present invention also relates to a Tpl2 kinase inhibitor or an inhibitor of the Tpl2 kinase gene expression for use in the prevention or treatment of instant blood-mediated inflammatory reaction (IBMIR) in a patient with a pancreatic β-cell transplant.

The invention thus embraces:

A method of inhibiting ERK1/2 and p90RSK activation (kinase activity) in a pancreatic β-cell in response to two or more pro-inflammatory cytokines by exposing said cell to Tpl2 kinase inhibitor. Said method may be performed in vitro, ex vivo or, in vivo. For example the β-cells may be present in a preparation of islet cells for transplantation.

Preferably the two or more pro-inflammatory cytokines are selected from: IL-1β, TNF-α and IFN-γ. Preferably the pro-inflammatory cytokines include at least IL-1β and. TNF-α. Preferably the Tpl2 kinase inhibitor is not used to completely inhibit Tpl2 kinase (for example by complete gene knockout). Partial inhibition is preferred e.g. sufficient to achieve 50 or 60% inhibition of the Tpl2 kinase activity in vivo. Furthermore the inhibition is “specifically” in response to two or more pro-inflammatory cytokines (for examples the physiological cytokines and chemokines secreted by inflammatory macrophages) but does not inhibit ERK1/2 and p90RSK activation (kinase activity) in response to glucose. Thus said specificity is of particular utility in providing a viable pharmaceutical intervention because it does not interfere with the role of ERK1/2 in glucose-mediated β-cell survival.

Thus, in embodiments of the invention, the inhibition of ERK1/2 and p90RSK activation (kinase activity) in a pancreatic β-cell in response to two or more pro-inflammatory cytokines is without impairing glucose-mediated survival of said β-cells.

Furthermore, in embodiments of the invention, the inhibition of ERK1/2 and p90RSK activation (kinase activity) in a pancreatic β-cell in response to two or more pro-inflammatory cytokines has the effect of inhibiting apoptosis of said pancreatic β-cells, which may for example be verified by analysis of cleaved caspase-3 and cleaved PARP levels in the call.

In these and other aspects the Tpl2 kinase inhibitor is optionally used in conjunction with and an anti-diabetic drug e.g. a glucagon-like peptide-1 (GLP-1) receptor agonist, e.g. an inhibitor of dipeptidyl peptidase 4 (DPP-4), the enzyme responsible for GLP-1 degradation, for example to protect said pancreatic β-cells in a patient suffering from, or at risk of, T2DM from cytokine-induced insulin secretion failure. As demonstrated by the experimental results herein, applying the inhibitors to subjects with a defined high risk for developing T2DM may slow the progression or even prevent T2DM in the subject.

Furthermore, in these and other aspects the Tpl2 kinase inhibitor is optionally used in anti-diabetic drug e.g. a glucagon-like peptide-1 (GLP-1) receptor agonist, for example to a pancreatic β-cell transplant from IBMIR in a recipient patient.

Thus the inhibitor and optionally drug can be used in vivo to ultimately improve glucose tolerance while reducing fasting blood glucose (i.e. inhibiting hyperglycemia) and serum insulin levels and\or improving or increasing insulin sensitivity of said cells. Preferably this is without effect on the body weight.

In other aspects of the invention, there are provided a Tumor Progression Locus-2 (Tpl2) kinase inhibitor for use in the methods described above, or a Tpl2 kinase inhibitor and an anti-diabetic drug for use in the methods described above.

In other aspects of the invention, there are provided use of a Tumor Progression Locus-2 (Tpl2) kinase inhibitor in the preparation of a medicament for use in the methods described above, or use of both a Tpl2 kinase inhibitor and an anti-diabetic drug in the preparation of such a medicament.

These and other aspects of the invention are discussed hereinafter.

### DETAILED DESCRIPTION OF THE INVENTION

As noted above, the invention is based on the discovery that the MAP3 kinase Tpl2 specifically mediates signaling pathways induced by inflammatory cytokines in β-cells, and plays an important role in triggering β-cell dysfunction and destruction and that Tpl2 kinase inhibitors protect pancreatic β-cells from apoptosis. Accordingly, Tpl2 kinase inhibitors are useful for preventing and treating diabetes and promoting β-cell survival in a number of applications.

The inventors have shown that the Tpl2 kinase is expressed in β-cells, mouse and human pancreatic islets, and is specifically involved in ERK1/2 activation by IL-1β alone or a cytokine mixture (IL-1β+TNFα+IFNγ) and have demonstrated that pharmacological inhibition of Tpl2 kinase prevents ERK1/2 activation and the detrimental effects of chronic exposure of IL-1β alone or of a cytokine mixture on β-cells and human pancreatic islets.

Moreover, the inventors have shown that Tpl2 kinase inhibitors are also useful for improving the anti-diabetic efficacy of a GLP-1 agonist (e.g. Exendin-4, liraglutide) of a DPP-4 inhibitor (e.g. Sitagliptin) by enhancing for instance their beneficial effects on pancreatic β-cell viability and function against pro-inflammatory cytokines They have shown that combination of Exendin-4, liraglutide, sitagliptin and inhibition of Tpl2 kinase more efficiently protects β-cells against the deleterious effect of inflammatory cytokines than each compound alone.

Based on the remarkable protective effects of Tpl2 kinase inactivation, they have found that inhibition of Tpl2 kinase significantly decreased cytokine-induced insulin secretion failure in human islets. Notably, human islets treated with combination of Tpl2 kinase inhibitor and Exendin-4 were found to be viable and functional, and totally protected against the detrimental effects of cytokines. Importantly, the use of Tpl2 kinase inhibitor enhances the protective effect of Exendin-4 against inflammation in β-cells and human islets.

### Therapeutic Methods and Uses

The present invention provides methods and compositions (such as pharmaceutical compositions) for preventing (e.g. prophylactic treatment) or treating diabetes.

In a first aspect, the present invention relates to a Tpl2 kinase inhibitor for use in the prevention or treatment of diabetes in a patient in need thereof.

As used herein, the term “Tumor Progression Locus-2 (Tpl2) kinase” refers to a serine/threonine kinase (also known as COT and MAP3K8) in the MAP3K family that is upstream of MEK1/2 in the ERK1/2 pathway which has been shown to be involved in both production and signaling of TNF-α. An exemplary native polynucleotide sequence encoding the human Tpl2 kinase is provided in GenBank database under accession number NM_{—}005204.

As used herein, the term “inhibitor” refers to any compound, natural or synthetic, which can reduce activity of a gene product. Accordingly, an inhibitor may inhibit the activity of a protein that is encoded by a gene either directly or indirectly. Direct inhibition can be obtained, for instance, by binding to a protein and thereby preventing the protein from binding a target (such as a binding partner) or preventing protein activity (such as enzymatic activity). Indirect inhibition can be obtained, for instance, by binding to a protein's intended target, such as a binding partner, thereby blocking or reducing activity of the protein.

As used herein, the term “Tpl2 kinase inhibitor” refers to any compound, natural or synthetic, which results in a decreased activity of Tpl2 kinase. Typically, an inhibitor of the Tpl2 kinase provokes a decrease in the levels of phosphorylation of the protein MEK and also an inhibition of TNF-α production in response to lipopolysaccharides (LPS) as described in Kaila et al., 2007.

The skilled person in the art can assess whether a given compound is a Tpl2 kinase inhibitor without undue burden. Typically, a compound is deemed to be a Tpl2 kinase inhibitor if, after carrying out a Tpl2 kinase enzymatic assay using MEK as a substrate in the presence of said compound, the level of phosphorylated MEK is decreased compared to MEK cultured in the absence of said compound. Levels of phosphorylated MEK1 proteins can be measured by Western blot or ELISA using antibodies specific for the phosphorylated form of said MEK1 proteins. For instance, Tpl2/Cot kinase activity may be directly assayed using GST-MEK1 as a substrate and the phosphorylation on serine residues 217 and 221 of GST-MEK1 may be detected by an ELISA as described in Kaila et al., 2007.

Additionally, inhibition of TNF-α by a given compound may be determined in vitro (e.g. in primary human monocytes or in human blood) or in vivo (e.g. a rat model of LPS-induced TNF-alpha production) as described in Kaila et al., 2007.

As used herein, “diabetes” refers to the broad class of metabolic disorders characterized by impaired insulin production and glucose tolerance. Diabetes includes type 1 and type 2 diabetes, gestational diabetes, prediabetes, insulin resistance, metabolic syndrome, impaired fasting glycaemia and impaired glucose tolerance. Type 1 diabetes is also known as Insulin Dependent Diabetes Mellitus (IDDM). The terms are used interchangeably herein. Type 2 is also known as Non-Insulin-Dependent Diabetes Mellitus (NIDDM).

As used herein, the term “a patient in need thereof” refers to a subject that has been diagnosed with type 1 diabetes, type 2 diabetes, gestational diabetes, pre-diabetes, insulin resistance, metabolic syndrome, impaired fasting glycaemia or impaired glucose tolerance, or one that is at risk of developing any of these disorders. Patients in need of treatment also include those that have suffered an injury, disease, or surgical procedure affecting the pancreas, or individuals otherwise impaired in their ability to make insulin. Such patients may be any mammal, e.g., human, dog, cat, horse, pig, sheep, bovine, mouse, rat or rabbit (preferably a human).

In one embodiment, the patient in need thereof is an obese patient.

The term “obesity” as used herein is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a BMI greater than or equal to 30 kg/m² An “obese patient” is an otherwise healthy subject with a BMI greater than or equal to 30 kg/m². An overweight subject is a subject at risk of obesity.

In another embodiment, the patient in need thereof is a lean patient.

Accordingly, a lean patient is an otherwise healthy subject with a BMI lesser than or equal to 25 kg/m² or even lesser or equal to 20 kg/m².

In one embodiment, the patient in need thereof is non-insulin resistant patient.

The term “preventing a disorder” as used herein, is not intended as an absolute term. Instead, prevention, e.g., of type 2 diabetes, refers to delay of onset, reduced frequency of symptoms, or reduced severity of symptoms associated with the disorder. Prevention therefore refers to a broad range of prophylactic measures that will be understood by those in the art. In some circumstances, the frequency and severity of symptoms is reduced to non-pathological levels, e.g., so that the individual does not need traditional insulin replacement therapy. In some circumstances, the symptoms of a patient receiving a Tpl2 kinase inhibitor according to the invention are only 90, 80, 70, 60, 50, 40, 30, 20, 10, 5 or 1% as frequent or severe as symptoms experienced by an untreated individual with the disorder.

Similarly, the term “treating a disorder” is not intended to be an absolute term. In some circumstances, the Tpl2 kinase inhibitors according to the invention seek to reduce the loss of insulin producing cells that lead to diabetic symptoms. In some circumstances, treatment with the inhibitors of the invention leads to an improved prognosis or a reduction in the frequency or severity of symptoms.

In one embodiment, the Tpl2 kinase may be a low molecular weight antagonist, e. g. a small organic molecule.

In a particular embodiment, such Tpl2 kinase inhibitors and their method of preparation are described in the international Patent Application WO 2006/124944 and have the following formula (I):

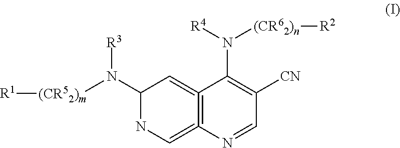

wherein:

R¹ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) N₃, e) OR⁷, f) NR⁸R⁹, g) oxo, h) thioxo, i) S(O)_{P}R⁷, j) SO₂NR⁸R⁹, k) C(O)R⁷, l) C(O)OR⁷, m) C(O)NR⁸R⁹, n) Si(C₁₋₆alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆haloalkyl, u) C₃₋₁₀ cycloalkyl, v) aryl, w) 3-10 membered cycloheteroalkyl, and x) heteroaryl, wherein any of o)-x) optionally is substituted with 1-4 R¹⁰ groups;
   R² is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) N₃, e) OR⁷, f) NR⁸R⁹, g) oxo, h) thioxo, i) S(O)_{P}R⁷, j) SO₂NR⁸R⁹, k) C(O)R⁷, l) C(O)OR⁷, m) C(O)NR⁸R⁹, n) Si(C₁₋₆alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl, wherein any of o)-x) optionally is substituted with 1-4 R¹⁰ groups;
   alternatively, R² is selected from the group consisting of halogen, C¹⁻⁶ alkyl optionally substituted with 1-4 R¹⁰ groups, C₁₋₆ haloalkyl, NR⁸R⁹, OR⁷, C(O)OR⁷, C(O)NR⁸R⁹, S(O)_{P}R⁷ and N₃;
   R³ and R⁴ independently are selected from the group consisting of:
a) H, b) C(O)R⁷, c) C(O)OR⁷, d) C(O)NR⁸R⁹, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and 1) heteroaryl;
wherein any of e)-l) optionally is substituted with 1-4 R¹⁰ groups;
   R⁵ and R⁶ at each occurrence independently are selected from the group consisting of:
a) H, b) halogen, c) OR⁷, d) NR⁸R⁹, e) C₁₋₆ alkyl, f) C₂₋₆ alkenyl, g) C₂₋₆ alkynyl, h) C₁₋₆ haloalkyl, and i) aryl;
   alternatively, any two R⁵ or R⁶ groups and the carbon to which they are bonded may form a carbonyl group;
   R⁷ at each occurrence is selected from the group consisting of:
a) H, b) C(O)R¹¹, c) C(O)OR¹¹, d) C(O)NR¹¹R¹², e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl;
wherein any of e)-l) optionally is substituted with 1-4 R¹³ groups;
   R⁸ and R⁹ at each occurrence independently are selected from the group consisting of:
a) H, b) OR¹¹, c) SO₂R¹¹, d) C(O)R¹¹, e) C(O)OR¹¹, f) C(O)NR¹¹R¹², g) C₁₋₆ alkyl, h) C₂₋₆ alkenyl, i) C₂₋₆ alkynyl, j) C₁₋₆ haloalkyl, k) C₃₋₁₀ cycloalkyl, 1) aryl, m) 3-10 membered cycloheteroalkyl, and n) heteroaryl;
   wherein any of g)-n) optionally is substituted with 1-4 R¹³ groups;
      R¹⁰ at each occurrence independently is selected from the group consisting of:
a) halogen, b) CN, c) NO₂, d) N₃, e) OR⁷, f) NR⁸R⁹, g) oxo, h) thioxo, i) S(O)_{P}R⁷, j) SO₂NR⁸R⁹, k) C(O)R⁷, l) C(O)OR⁷, m) C(O)NR⁸R⁹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl,
wherein any of o)-x) optionally is substituted with 1-4 R¹³ groups;
   R¹¹ and R¹² at each occurrence independently are selected from the group consisting of:
a) H b) C₁₋₆ alkyl, c) C₂₋₆ alkenyl, d) C₂₋₆ alkynyl, e) C₁₋₆ haloalkyl, f) C₃₋₁₀ cycloalkyl, g) aryl, h) 3-10 membered cycloheteroalkyl, and i) heteroaryl,
wherein any of b)-i) optionally is substituted with 1-4 R¹³ groups;
   R¹³ at each occurrence independently is selected from the group consisting of:
a) halogen, b) CN, c) NO₂, d) N₃, e) OH, f) O—C₁₋₆ alkyl, g) NH₂, h) NH(C₁₋₆ alkyl), i) N(C₁₋₆ alkyl)₂, j) NH(aryl), k) NH(cycloalkyl), l) NH(heteroaryl), m) NH(cycloheteroalkyl), n) oxo, o) thioxo, p) SH, q) S(O)ₚ—C₁₋₆ alkyl, r) C(O)—C₁₋₆ alkyl, s) C(O)OH, t) C(O)O—C₁₋₆ alkyl, u) C(O)NH₂, v) C(O)NHC₁₋₆ alkyl, w) C(O)N(C₁₋₆ alkyl)₂, x) C₁₋₆ alkyl, y) C₂₋₆ alkenyl, z) C₂₋₆ alkynyl, aa) C₁₋₆ alkoxy, bb) C₁₋₆ alkylthio, cc) C₁₋₆ haloalkyl, dd) C₃₋₁₀ cycloalkyl, ee) aryl, ff) 3-10 membered cycloheteroalkyl, and gg) heteroaryl,
   wherein any C₁₋₆ alkyl, C₂₋₆ alkenyl, C₂₋₆ alkynyl, C₃₋₁₀ cycloalkyl, aryl, 3-10 membered cycloheteroalkyl, or heteroaryl, alone as a part of another moiety, optionally is substituted with one or more moieties selected from the group consisting of halogen, CN, NO₂, OH, O—C₁₋₆ alkyl, NH₂, NH(C₁₋₆ alkyl), N(_{C1-6} alkyl)₂, NH(aryl), NH(cycloalkyl), NH(heteroaryl), NH(cycloheteroalkyl), oxo, thioxo, SH, S(O)ₚ—C₁₋₆ alkyl, C(O)—C₁₋₆ alkyl, C(O)OH, C(O)O—C₁₋₆ alkyl, C(O)NH₂, C(O)NHC₁₋₆ alkyl, C(O)N(C₁₋₆ alkyl)₂, C₁₋₆ alkyl, C₂₋₆ alkenyl, C₂₋₆ alkynyl, C₁₋₆ alkoxy, C1-6 alkylthio, C₁₋₆ haloalkyl, C₃₋₁₀ cycloalkyl, aryl, 3-10 membered cycloheteroalkyl, and heteroaryl;
      m is 0, 1, 2, 3, or 4;
      n is 0 or 1; and
      p is 0, 1, or 2;
      or a pharmaceutically acceptable salt thereof.

In a preferred embodiment, such Tpl2 kinase inhibitor is 4-(3-cloro-4-fluorophenylamino)-6-(pyridine-3-yl-methylamino)-3-cyano-[1,7]-napthyridine having the following formula:

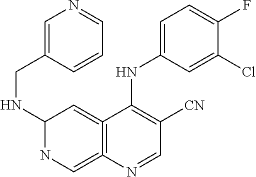

In another preferred particular embodiment, such Tpl2 kinase inhibitor is 4-cycloheptylamino-6-[(pyridin-3-ylmethyl)-amino]-[1,7]naphthyridine-3-carbonitrile as described in Kaila et al., 2007 having the following formula:

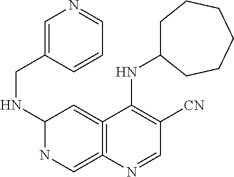

In another particular embodiment, such Tpl2 kinase inhibitors and their method of preparation are described in the international Patent Application WO 2006/124692 and have the following formula (II):

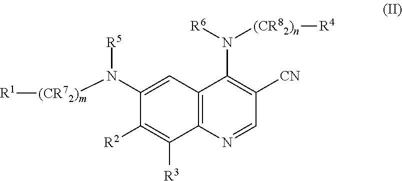

wherein:

R¹ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) N₃, e) OR⁹, f) NR¹⁰R¹¹, g) oxo, h) thioxo, i) S(O)PR⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl,
wherein any of o)-x) optionally is substituted with 1-4 R¹² groups;
   R² is selected from the group consisting of:
a) H, b) halogen, c) CN, d) NO₂, e) OR⁹, f) NR¹⁰R¹¹, g) S(O)PR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₃₋₁₀ cycloalkyl, r) aryl, s) 3-10 membered cycloheteroalkyl, and t) heteroaryl, wherein any of l)-t) optionally is substituted with 1-4 R¹² groups;
   R³ is selected from the group consisting of:
a) H, b) halogen, c) CN, d) NO₂, e) OR⁹, f) NR¹⁰R¹¹, g) S(O)PR⁹, h) SO₂NR¹⁰R¹¹, i) C(O)R⁹, j) C(O)OR⁹, k) C(O)NR¹⁰R¹¹, l) C₁₋₆ alkyl, m) C₂₋₆ alkenyl, n) C₂₋₆ alkynyl, o) C₁₋₆ alkoxy, p) C₁₋₆ alkylthio, q) C₁₋₆ haloalkyl, r) C₃₋₁₀ cycloalkyl, s) aryl, t) 3-10 membered cycloheteroalkyl, and u) heteroaryl,
wherein any of l)-u) optionally is substituted with 1-4 R¹² groups;
   R⁴ is selected from the group consisting of C₃₋₁₀ cycloalkyl, aryl, 3-10 membered cycloheteroalkyl, and heteroaryl, each optionally substituted with 1-4 moieties selected from the group consisting of:
a) halogen, b) CN, c) NO₂, d) OR⁹, e) NR¹⁰R¹¹, f) oxo, g) thioxo, h) S(O)PR⁹, i) SO₂NR¹⁰R¹¹, j) C(O)R⁹, k) C(O)OR⁹, l) C(O)NR¹⁰R¹¹, m) Si(C₁₋₆ alkyl)₃, n) C₁₋₆ alkyl, o) C₂₋₆ alkenyl, p) C₂₋₆ alkynyl, q) C₁₋₆ alkoxy, r) C₁₋₆ alkylthio, s) C₁₋₆ haloalkyl, t) C₃₋₁₀ cycloalkyl, u) aryl, v) 3-10 membered cycloheteroalkyl, and w) heteroaryl,
wherein any of n)-w) optionally is substituted with 1-4 R¹² groups;
   alternatively, R⁴ is selected from the group consisting of C₁₋₆ alkyl optionally substituted with 1-4 R¹² groups, C₁₋₆ haloalkyl, OR⁹, NR¹⁰R¹¹, C(O)OR⁹, C(O)NR¹⁰R¹¹, S(O)ₚR⁹, and N₃;
   R⁵ and R⁶ at each occurrence independently are selected from the group consisting of:
a) H, b) C(O)R⁹, c) C(O)OR9, d) C(O)NR¹⁰R¹¹, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl,
wherein any of e)-l) optionally is substituted with 1-4 R¹² groups;
   R⁷ and R⁸ at each occurrence independently are selected from the group consisting of:
a) H, b) halogen, c) OR⁹, d) NR¹⁰R¹¹, e) C₁₋₆ alkyl, f) C₂₋₆ alkenyl, g) C₂₋₆ alkynyl, h) C₁₋₆ haloalkyl, and i) aryl;
   alternatively, any two R⁷ or R⁸ groups and the carbon to which they are bonded may form a carbonyl group;
   R⁹ at each occurrence is selected from the group consisting of:
a) H, b) C(O)R¹³, c) C(O)OR¹³, d) C(O)NR¹³R¹⁴, e) C₁₋₆ alkyl, f) C₂₋₆ alkenyl, g) C₂₋₆ alkynyl, h) C₁₋₆ haloalkyl, i) C₃₋₁₀ cycloalkyl, j) aryl, k) 3-10 membered cycloheteroalkyl, and l) heteroaryl, wherein any of e)-l) optionally is substituted with 1-4 R¹⁵ groups;
   R¹⁰ and R¹¹ at each occurrence independently are selected from the group consisting of:
a) H, b) OR¹³, c) SO₂R¹³, d) C(O)R¹³, e) C(O)OR¹³, f) C(O)NR¹³R¹⁴, g) C₁₋₆ alkyl, h) C₂₋₆ alkenyl, i) C₂₋₆ alkynyl, k) C₁₋₆ haloalkyl, I) C₃₋₁₀ cycloalkyl, m) aryl, n) 3-10 membered cycloheteroalkyl, and o) heteroaryl;
wherein any of g)-o) optionally is substituted with 1-4 R¹⁵ groups;
   R¹² at each occurrence independently is selected from the group consisting of:
a) halogen, b) CN, c) NO₂, d) N₃, e) OR⁹, f) NR¹⁰R¹¹, g) oxo, h) thioxo, i) S(O)_{P}R⁹, j) SO₂NR¹⁰R¹¹, k) C(O)R⁹, l) C(O)OR⁹, m) C(O)NR¹⁰R¹¹, n) Si(C₁₋₆ alkyl)₃, o) C₁₋₆ alkyl, p) C₂₋₆ alkenyl, q) C₂₋₆ alkynyl, r) C₁₋₆ alkoxy, s) C₁₋₆ alkylthio, t) C₁₋₆ haloalkyl, u) C₃₋₁₀ cycloalkyl, v) aryl, w) 3-10 membered cycloheteroalkyl, and x) heteroaryl;
   wherein any of o)-x) optionally is substituted with 1-4 R¹⁵ groups;
      R¹³ and R¹⁴ at each occurrence independently are selected from the group consisting of:
a) H, b) C₁₋₆ alkyl, c) C₂₋₆ alkenyl, d) C₂₋₆ alkynyl, e) C₁₋₆ haloalkyl, f) C₃₋₁₀ cycloalkyl, g) aryl, h) 3-10 membered cycloheteroalkyl, and i) heteroaryl,
wherein any of b)-j) optionally is substituted with 1-4 R¹⁵ groups;
   R¹⁵ at each occurrence independently is selected from the group consisting of:
a) halogen, b) CN, c) NO₂, d) N₃, e) OH, f) 0-C₁₋₆ alkyl, g) NH₂, h) NH(C₁₋₆ alkyl), i) N(C₁₋₆ alkyl)₂, j) NH(aryl), k) NH(cycloalkyl), l) NH(heteroaryl), m) NH(cycloheteroalkyl), n) oxo, o) thioxo, p) SH, q) S(O)_{P}—C₁₋₆ alkyl, r) C(O)—C₁₋₆ alkyl, s) C(O)OH, t) C(O)O—C₁₋₆ alkyl, u) C(O)NH₂, v) C(O)NHC₁₋₆ alkyl, w) C(O)N(C₁₋₆ alkyl)₂, x) C₁₋₆ alkyl, y) C₂₋₆ alkenyl, z) C₂₋₆ alkynyl, aa) C₁₋₆ alkoxy, bb) Ci-6 alkylthio, cc) C₁₋₆ haloalkyl, dd) C₃₋₁₀ cycloalkyl, ee) aryl, ff) 3-10 membered cycloheteroalkyl, and gg) heteroaryl,
   wherein any C₁₋₆ alkyl, C₂₋₆ alkenyl, C₂₋₆ alkynyl, C₃₋₁₀ cycloalkyl, aryl, 3-10 membered cycloheteroalkyl, or heteroaryl, alone as a part of another moiety, optionally is substituted with one or more moieties selected from the group consisting of halogen, CN, NO₂, OH, O—C₁₋₆ alkyl, NH₂, NH(C₁₋₆ alkyl), N(C₁₋₆ alkyl)₂, NH(aryl), NH(cycloalkyl), NH(heteroaryl), NH(cycloheteroalkyl), oxo, thioxo, SH, S(O)ₚ—C₁₋₆ alkyl, C(O)—C₁₋₆ alkyl, C(O)OH, C(O)O—C₁₋₆ alkyl, C(O)NH₂, C(O)NHC₁₋₆ alkyl, C(O)N(C₁₋₆ alkyl)₂, C₁₋₆ alkyl, C₂₋₆ alkenyl, C₂₋₆ alkynyl, C₁₋₆ alkoxy, C₁₋₆ alkylthio, C₁₋₆ haloalkyl, C₃₋₁₀ cycloalkyl, aryl, 3-10 membered cycloheteroalkyl, and heteroaryl;
      m is 0, 1, 2, 3, or 4;
      n is 0 or 1; and
      p is 0, 1, or 2;
      or a pharmaceutically acceptable salt thereof.

In a particular embodiment, such Tpl2 kinase inhibitor is 8-chloro-4-(3-chloro-4-fluorophenylamino)-6-((1-(1-ethylpiperidin-4-yl)-1H-1,2,3-triazol-4-yl)methylamino) quinoline-3-carbonitrile as described in Wu et al., 2009 having the following formula:

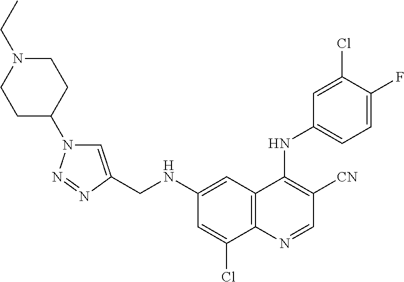 [SMILES: CCN1CCC(n2cc(CNc3cc(Cl)c4ncc(C#N)c(Nc5ccc(F)c(Cl)c5)c4c3)nn2)CC1]

Other Tpl2 kinase inhibitors are described in the international Patent Applications WO/001191 and WO 2005/110410 and in George and Salmeron, 2009.

In a second aspect, the present invention relates to an inhibitor of Tpl2 kinase gene expression for use in the prevention or treatment of diabetes in a patient in need thereof.

As used herein, the term “inhibitor of gene expression” refers to a natural or synthetic compound that has a biological effect to inhibit or significantly reduce the expression of a gene. Consequently an “inhibitor of Tpl2 kinase gene expression” refers to a natural or synthetic compound that has a biological effect to inhibit or significantly reduce the expression of the gene encoding for the Tpl2 kinase.

Inhibitors of Tpl2 kinase gene expression for use in the present invention may be based on anti-sense oligonucleotide constructs. Anti-sense oligonucleotides, including anti-sense RNA molecules and anti-sense DNA molecules, would act to directly block the translation of Tpl2 kinase mRNA by binding there to and thus preventing protein translation or increasing mRNA degradation, thus decreasing the level of Tpl2 kinase, and thus activity, in a cell. For example, antisense oligonucleotides of at least about 15 bases and complementary to unique regions of the mRNA transcript sequence encoding Tpl2 kinase can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of Tpl2 kinase gene expression for use in the present invention. Tpl2 kinase gene expression can be reduced by contacting a subject or cell with a small double stranded RNA (dsRNA), or a vector or construct causing the production of a small double stranded RNA, such that Tpl2 kinase gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In one embodiment, Tpl2 kinase gene expression may be inhibited by using a validated set of 4 different 19-nucleotides siRNA duplexes (“ON-TARGETplus SMARTpool”, L-091828-01-0005) purchased from Dharmacon (ABgene Ltd, part of Thermo Fisher Scientific, Waltham, Mass.) as described in the section EXAMPLES below.

Ribozymes can also function as inhibitors of Tpl2 kinase gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of Tpl2 kinase mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of Tpl2 kinase gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing Tpl2 kinase. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

The present invention further relates to a Tpl2 kinase inhibitor or an inhibitor of the Tpl2 kinase gene expression for use in improving survival or function of pancreatic β-cells in a patient in need thereof.

The present invention also relates to a method for preventing or treating diabetes comprising administering to a patient in need thereof a Tpl2 kinase inhibitor or an inhibitor of Tpl2 kinase gene expression.

Tpl2 kinase inhibitors or inhibitors of Tpl2 kinase gene expression may be administered in the form of a pharmaceutical composition, as defined below. Preferably, said antagonist or inhibitor is administered in a therapeutically effective amount.

By a “therapeutically effective amount” is meant a sufficient amount of the Tpl2 kinase inhibitor or inhibitor of Tpl2 kinase gene expression to prevent or treat diabetes at a reasonable benefit/risk ratio applicable to any medical treatment.

It will be understood that the total daily use of the compounds of the present invention will be decided by the attending physician within the scope of medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions

The Tpl2 kinase inhibitor or inhibitor of Tpl2 kinase gene expression may be combined with pharmaceutically acceptable excipients, and optionally sustained-release matrices, such as biodegradable polymers, to form therapeutic compositions.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The Tpl2 kinase inhibitor or inhibitor of Tpl2 kinase gene expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The Tpl2 kinase inhibitor or inhibitor of Tpl2 kinase gene expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may comprise an additional therapeutic active agent.

In one embodiment, said additional therapeutic active agent is an anti-diabetic drug as described below.

In a particular aspect, the invention also relates to a pharmaceutical composition for use in improving survival or function of pancreatic β-cells in a patient in need thereof as above described.

### A Kit-of-Part for Use in the Prevention or Treatment of Diabetes

The Tpl2 kinase inhibitor or inhibitor of Tpl2 kinase gene expression of the invention may also be used in combination with other therapeutically active agents, for instance, an anti-diabetic drug (e.g. a glucagon-like peptide-1 (GLP-1) receptor agonist).

In one embodiment, the Tpl2 kinase inhibitor or inhibitor of Tpl2 kinase gene expression or a pharmaceutical composition comprising thereof may be intended to be administered separately, sequentially or simultaneously with an anti-diabetic drug.

More particularly, in the “combination” treatments described herein two or more treatments or therapies are combined, for example, sequentially or simultaneously. The agents may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g. 1, 2, 3, 4 or more hours apart, or even longer periods apart where required), the precise dosage regimen being commensurate with the properties of the therapeutic agent(s) as described herein, including their synergistic effect.

The agents may be formulated together in a single dosage form, or alternatively, the individual agents may be formulated separately and presented together in the form of a kit (e.g. in blister packs) optionally with instructions for their use.

Accordingly, in a third aspect, the present invention also relates to a kit-of-part that is suitable for use in the prevention or treatment of diabetes comprising a Tpl2 kinase inhibitor or an inhibitor of the Tpl2 kinase gene expression and an anti-diabetic drug.

In one embodiment, the kit-of-part of the invention may comprise (i) a Tpl2 kinase inhibitor or an inhibitor of the Tpl2 kinase gene expression, as defined above, and (ii) at least one anti-diabetic drug, each of (i) and (ii) being laid out to be administered separately, sequentially or simultaneously.

As used herein, the term “anti-diabetic drug” refers to any compound, natural or synthetic, which can reduce glucose levels in the blood and therefore is useful for preventing or treating diabetes. Typically, anti-diabetic drugs encompass (1) insulin as well as insulin analogs (e.g. insulin lispro marketed by Eli Lilly as “Humalog”) or variants, (2) agents that increase the amount of insulin secreted by the pancreas (e.g. glucagon-like peptide-1 (GLP-1) receptor agonists, DPP-4 inhibitors, and sulfonylureas) (3) agents that increase the sensitivity of target organs to insulin (e.g. biguanides and thiazolidinediones), and (4) agents that decrease the rate at which glucose is absorbed from the gastrointestinal tract (e.g. alpha-glucosidase inhibitors).

In one particular embodiment, the anti-diabetic drug is insulin. Human insulin is a 51 amino acid peptide hormone produced in the islets of Langerhans in the pancreas.

In another particular embodiment, the anti-diabetic drug is an insulin analog or variant.

Human insulin has three primary amino groups: the N-terminal group of the A-chain and of the B-chain and the ε-amino group of Lys^{B29}. Several insulin analogs or variants which are substituted in one or more of these groups are known in the prior art as described in WO2007/074133. Exemplary insulin analogs that are contemplated by the invention include insulin modified at amino acid position 29 of the native human insulin B chain and optionally at other positions. For instance, a preferred analog of insulin is insulin lispro marketed by Eli Lilly as “Humalog” and described in U.S. Pat. No. 5,514,646. Such insulin analog is one wherein B28 is lysine and B29 is proline, i.e., an inversion of the native human insulin amino acid sequence at positions 28 and 29 of the B-chain.

The insulin analogs of this invention can be prepared by any of a variety of recognized peptide synthesis techniques including classical (solution) methods, solid-phase methods, semi synthetic methods and the more recently available recombinant DNA methods.

In one particular embodiment, the anti-diabetic drug is a glucagon-like peptide-1 (GLP-1) receptor agonist.

Exemplary GLP-1 receptor agonists that are contemplated by the invention include but are not limited to exenatide or specific formulations thereof, as described, for example, in WO2008061355, WO2009080024, WO2009080032, liraglutide, taspoglutide (R-1583), albiglutide, lixisenatide or those which have been disclosed in WO 98/08871, WO2005027978, WO2006037811, WO2006037810 by Novo Nordisk A/S, in WO 01/04156 by Zealand or in WO 00/34331 by Beaufour-Ipsen, pramlintide acetate (Symlin; Amylin Pharmaceuticals), inhalable GLP-1 (MKC-253 from MannKind) AVE-0010, BIM-51077 (R-1583, ITM-077), PC-DAC:exendin-4 (an exendin-4 analog which is bonded covalently to recombinant human albumin), biotinylated exendin (WO2009107900), a specific formulation of exendin-4 as described in US2009238879, CVX-73, CVX-98 and CVx-96 (GLP-1 analogs which are bonded covalently to a monoclonal antibody which has specific binding sites for the GLP-1 peptide), CNTO-736 (a GLP-1 analog which is bonded to a domain which includes the Fc portion of an antibody), PGC-GLP-1 (GLP-1 bonded to a nanocarrier), agonists or modulators, as described, for example, in D. Chen et al., Proc. Natl. Acad. Sci. USA 104 (2007) 943, those as described in WO2006124529, WO2007124461, WO2008062457, WO2008082274, WO2008101017, WO2008081418, WO2008112939, WO2008112941, WO2008113601, WO2008116294, WO2008116648, WO2008119238, WO2008148839, US2008299096, WO2008152403, WO2009030738, WO2009030771, WO2009030774, WO2009035540, WO2009058734, WO2009111700, WO2009125424, WO2009129696, WO2009149148, peptides, for example obinepitide (TM-30338), orally active GLP-1 analogs (e.g. NN9924 from Novo Nordisk), amylin receptor agonists, as described, for example, in WO2007104789, WO2009034119, analogs of the human GLP-1, as described in WO2007120899, WO2008022015, WO2008056726, chimeric pegylated peptides containing both GLP-1 and glucagon residues, as described, for example, in WO2008101017, WO2009155257, WO2009155258, glycosylated GLP-1 derivatives as described in WO2009153960, and orally active hypoglycemic ingredients.

In a preferred embodiment, the GLP-1 receptor agonist is exendin-4 or exenatide.

Exendin-4 is described in the U.S. Pat. No. 5,424,286 and is a hormone found in the saliva of the Gila monster which displays biological properties similar to human glucagon-like peptide-1 (GLP-1), a regulator of glucose metabolism and insulin secretion.

Exenatide is a 39-amino-acid peptide and a synthetic version of exendin-4, which enhances glucose-dependent insulin secretion by the pancreatic β-cell and suppresses inappropriately elevated glucagon secretion.

In another preferred embodiment, the GLP-1 receptor agonist is liraglutide.

In another particular embodiment, the anti-diabetic drug is an inhibitor of dipeptidyl peptidase-IV (DDP-4).

Exemplary inhibitors of DDP-4 that are contemplated by the invention include but are not limited to vildagliptin (LAF-237), sitagliptin (MK-0431), sitagliptin phosphate, saxagliptin (BMS-477118), GSK-823093, PSN-9301, SYR-322, SYR-619, TA-6666, TS-021, GRC-8200 (melogliptin), GW-825964X, KRP-104, DP-893, ABT-341, ABT-279 or another salt thereof, S-40010, S-40755, PF-00734200, BI-1356, PHX-1149, DSP-7238, alogliptin benzoate, linagliptin, melogliptin, carmegliptin, or those compounds as described in WO2003074500, WO2003106456, WO2004037169, WO200450658, WO2005037828, WO2005058901, WO2005012312, WO2005/012308, WO2006039325, WO2006058064, WO2006015691, WO2006015701, WO2006015699, WO2006015700, WO2006018117, WO2006099943, WO2006099941, JP2006160733, WO2006071752, WO2006065826, WO2006078676, WO2006073167, WO2006068163, WO2006085685, WO2006090915, WO2006104356, WO2006127530, WO2006111261, US2006890898, US2006803357, US2006303661, WO2007015767 (LY-2463665), WO2007024993, WO2007029086, WO2007063928, WO2007070434, WO2007071738, WO2007071576, WO2007077508, WO2007087231, WO2007097931, WO2007099385, WO2007100374, WO2007112347, WO2007112669, WO2007113226, WO2007113634, WO2007115821, WO2007116092, US2007259900, EP1852108, US2007270492, WO2007126745, WO2007136603, WO2007142253, WO2007148185, WO2008017670, US2008051452, WO2008027273, WO2008028662, WO2008029217, JP2008031064, JP2008063256, WO2008033851, WO2008040974, WO2008040995, WO2008060488, WO2008064107, WO2008066070, WO2008077597, JP2008156318, WO2008087560, WO2008089636, WO2008093960, WO2008096841, WO2008101953, WO2008118848, WO2008119005, WO2008119208, WO2008120813, WO2008121506, WO2008130151, WO2008131149, WO2009003681, WO2009014676, WO2009025784, WO2009027276, WO2009037719, WO2009068531, WO2009070314, WO2009065298, WO2009082134, WO2009082881, WO2009084497, WO2009093269, WO2009099171, WO2009099172, WO2009111239, WO2009113423, WO2009116067, US2009247532, WO2010000469, WO2010015664.

In a preferred embodiment, the inhibitor of DDP-4 is sitagliptin.

It should be further noted that the inhibitor of DDP-4 may be administered in combination with metformin hydrochloride (e.g. Janumet®, a solid combination of sitagliptin phosphate with metformin hydrochloride or Eucreas®, a solid combination of vildagliptin with metformin hydrochloride).

In still another particular embodiment, the anti-diabetic drug is a GPR40 receptor agonist.

Exemplary of a GPR40 receptor agonists that are contemplated by the invention include but are not limited to those described, for example, in WO2007013689, WO2007033002, WO2007106469, US2007265332, WO2007123225, WO2007131619, WO2007131620, WO2007131621, US2007265332, WO2007131622, WO2007136572, WO2008001931, WO2008030520, WO2008030618, WO2008054674, WO2008054675, WO2008066097, US2008176912, WO2008130514, WO2009038204, WO2009039942, WO2009039943, WO2009048527, WO2009054479, WO2009058237, WO2009111056, WO2010012650, WO2011161030, WO2012004269, WO2012010413.

In a preferred embodiment, the GPR40 receptor agonist is TAK-875 or AMG 837.

In a further particular embodiment, the anti-diabetic drug is a thiazolidinedione, for example troglitazone, ciglitazone, pioglitazone, rosiglitazone or the compounds disclosed in WO 97/41097 by Dr. Reddy's Research Foundation, especially 5-[[4-[3,4-dihydro-3-methyl-4-oxo-2-quinazolinylmethoxy]-phenyl]methyl]-2,4-thiazolidinedione.

In a further particular embodiment, the anti-diabetic drug is a biguanide, for example metformin or one of its salts.

Other anti-diabetic drugs that are contemplated by the invention include but are not limited to those described, for example, in US 2012/0004166.

The present invention also relates to a kit-of-part for use in the prevention or treatment of diabetes comprising a Tpl2 kinase inhibitor or an inhibitor of the Tpl2 kinase gene expression and an anti-diabetic drug.

The present invention also relates to a method for preventing or treating diabetes comprising administering to a patient in need thereof a kit-of-part comprising a Tpl2 kinase inhibitor or an inhibitor of the Tpl2 kinase gene expression and an anti-diabetic drug.

The present invention further relates to the use of a Tpl2 kinase inhibitor or an inhibitor of the Tpl2 kinase gene expression for enhancing the clinical efficacy of an anti-diabetic drug. As used herein, the term “enhancing the clinical efficacy” refers to an improvement of the anti-inflammatory action and/or preserving pancreatic β-cell viability and function.

### A Culture Medium Comprising a Tpl2 Kinase Inhibitor

In a fourth aspect, the present invention further relates to a culture medium comprising a Tpl2 kinase inhibitor or an inhibitor of the Tpl2 kinase gene expression as defined above.

As used herein, the term “culture medium” refers to a liquid medium suitable for the in vitro or ex vivo culture of mammalian pancreatic β-cells, and preferably human pancreatic β-cells.

As used herein, “pancreatic β-cell”, “β islet cells”, “insulin producing cells” and similar terms refer a population of pancreatic endocrine cells found in the islets of Langerhans. β islet cells produce and secrete insulin and amylin into the bloodstream.

The culture medium used by the invention may be a water-based medium that includes a combination of substances such as salts, nutrients, minerals, vitamins, amino acids, nucleic acids, proteins such as cytokines, growth factors and hormones, all of which are needed for cell survival.

For example, a culture medium according to the invention may be a synthetic tissue culture medium such as the RPMI (Roswell Park Memorial Institute medium) or the CMRL-1066 (Connaught Medical Research Laboratory) for human use, supplemented with the necessary additives as is further described below (Section Examples).

For instance, after isolation, human islets are cultured in CMRL (Connaught Medical Research Laboratories) 1066 medium (purchased from Sigma-Aldrich (C0422)) comprising 5.6 mmol/l glucose and supplemented with 10% fetal bovine serum (FBS) or human serum albumin (HSA), 100 UI/ml penicillin, 100 mg/ml streptomycin and 2 mM glutamine.

In a preferred embodiment, the culture medium of the invention is free of animal-derived substances. In a preferred embodiment, the culture medium of the invention consists essentially of synthetic compounds, compounds of human origin and water. Advantageously, said culture medium can be used for culturing cells according to good manufacturing practices (under “GMP” conditions).

In a preferred embodiment, the Tpl2 kinase inhibitor is 4-(3-cloro-4-fluorophenylamino)-6-(pyridine-3-yl-methylamino)-3-cyano-[1,7]-napthyridine (which can be purchased from Calbiochem). Typically, said Tpl2 kinase inhibitor is added to the culture medium of the invention in a concentration ranging from 1 to 20 μM, preferably ranging from 2 to 10 μM, even more preferably at about 3 μM.

In one embodiment the culture medium comprises one or more isolated pancreatic endocrine cells found e.g. β cells as described above.

### Transplantation of Pancreatic β-Cells

In a fifth aspect, the present invention relates to a method for improving survival and/or function of a population of pancreatic β-cells in vitro or ex vivo, said method comprising a step of contacting said population with a culture medium comprising an effective amount of a Tpl2 kinase inhibitor or an inhibitor of the Tpl2 kinase gene expression as defined above.

As used herein, “improving cell survival” refers to an increase in the number of cells that survive a given condition, as compared to a control, e.g., the number of cells that would survive the same conditions in the absence of treatment. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition. In some embodiments, cell survival is improved by 5, 10, 20, 30, 40, 50, 60, 70, 80, 90 or 100%, as compared to control levels. In some embodiments, cell survival is by two-, three-, four-, five-, or ten-fold of control levels. Alternatively, improved cell survival can be expressed as a percentage decrease in apoptosis. In some embodiments, for example, apoptosis is reduced by 10, 20, 30, 40, 50, 60, 70, 80, 90 or up to 100%, as compared to a control sample.

The invention also relates to a method of preventing or reducing inflammation and/or apoptosis of a population of pancreatic β-cells in vitro or ex vivo, said method comprising a step of contacting said population with a culture medium comprising an effective amount of a Tpl2 kinase inhibitor or an inhibitor of the Tpl2 kinase gene expression as defined above.

In a further aspect, the present invention relates to a method for improving survival and/or function of a pancreatic β-cell transplant, said method comprising a step of pre-culturing the pancreatic β-cell transplant with a culture medium comprising an effective amount of Tpl2 kinase inhibitor or an inhibitor of the Tpl2 kinase gene expression as defined above.

In a further aspect, the present invention relates to a method for improving survival and/or function of a pancreatic β-cell transplant, said method comprising a step of administering an effective amount of Tpl2 kinase inhibitor or an inhibitor of the Tpl2 kinase gene expression as defined above to said patient with a pancreatic β-cell transplant.

In one embodiment, the Tpl2 kinase inhibitor or an inhibitor of the Tpl2 kinase gene expression is administrated to the patient (recipient) in the very first phase of transplantation.

In a further aspect, the present invention relates to a method for improving survival and/or function of a pancreatic β-cell transplant, said method comprising a first step of pre-culturing the pancreatic β-cell transplant with a culture medium comprising an effective amount of the Tpl2 kinase inhibitor or an inhibitor of the Tpl2 kinase gene expression as defined above and a second step of administering an effective amount of Tpl2 kinase inhibitor or an inhibitor of the Tpl2 kinase gene expression as defined above to said patient with the pre-cultured pancreatic β-cell transplant. A “transplant,” as used herein, refers to the introduction of cells into an individual (recipient or host). A “pancreatic β-cell transplant” refers to a transplant that includes β-cells, but is not necessarily composed entirely of β-cells.

In a further aspect, there is provided a population of isolated pancreatic β-cells for transplantation, wherein said cells have been treated after isolation and\or are in the presence of an exogenous Tpl2 kinase inhibitor.

The transplanted cells can be introduced as an entire organ (e.g., a pancreas), a largely intact tissue sample (e.g., a tissue graft, like islet transplantation), or as a disaggregated population of cells (e.g., enriched for β-islet cells) or a transplant of purified β-cells. The introduced cells can be from another individual (allotransplantation) or from the same individual (autotransplantation). In some cases, cells are removed from an individual, cultured under favorable conditions, and replaced. In some cases, undifferentiated or partially differentiated cells can be cultured under appropriate conditions to differentiate into β-cells, and transplanted into an individual.

In still a further aspect, the present invention relates to a Tpl2 kinase inhibitor or an inhibitor of the Tpl2 kinase gene expression as defined above for use in the prevention or treatment of instant blood-mediated inflammatory reaction (IBMIR) in a patient with a pancreatic β-cell transplant.

The present invention also relates to a method for preventing or treating IBMIR in a patient with a pancreatic β-cell transplant, said method comprising a step of administering an effective amount of Tpl2 kinase inhibitor or an inhibitor of the Tpl2 kinase gene expression as defined above to said patient with a pancreatic β-cell transplant.

The invention will be further illustrated by the following figures and examples. However, these examples and figures should not be interpreted in any way as limiting the scope of the present invention.

### FIGURES

FIG. 1: Tpl2 is expressed and activated by IL-1β and cytokines in β-cells. A: Proteins from lysates were prepared from INS-1E cells, or mouse or human islets. Lysates were subjected to Western blotting using Tpl2 antibody (1:250). B and C: INS-1E β-cells were stimulated in KRB buffer for the indicated times with IL-1β alone (20 ng/ml) (B) or a cytokine mixture (IL-1β (0.2 ng/ml), TNFα (50 ng/ml), and IFNγ (30 ng/ml) (C). Lysates were subjected to Western blotting with antibodies against Tpl2 (1:250) or β-actin (1:5000). Dotted line represents 100% of protein amount from untreated control cells. Representative immunoblots and quantification of four independent experiments are shown. Data are expressed as a percentage of Tpl2 protein amount in untreated cells and presented as the means±SEM. *P<0.05, and **P<0.01 vs untreated cells.

FIGS. 2 and 3: Pharmacological inhibition or silencing of Tpl2 specifically prevents ERK1/2 and p90RSK activation in response to IL-1β and cytokines in INS-1E β-cells. 2A, 2B, 2C, 3A and 3C: INS-1E β-cells (2A, 2B, 2C and 3A) or mouse islets (3C) were treated in KRB buffer with or without Tpl2 inhibitor (Tpl2-I) (3 μM) during 2 h and then stimulated or not with IL-1β alone (20 ng/ml) (2A and 2B), a cytokine mix (IL-1β (0.2 ng/ml), TNF-α (50 ng/ml), and IFN-γ (30 ng/ml) (2C), or glucose (10 mM) (3A) for 20 min. Lysates were subjected to Western blotting with antibodies against Tpl2 (1:250), phosphorylated or total ERK1/2 (1:2000), phosphorylated or total p38 (1:1000), phosphorylated or total p54/p46 JNK (1:1000) or phosphorylated p90RSK (1:1000). Quantification of four or five independent experiments is shown. 2D and 3B: 72 h after the first 40 nM siRNA transfection, INS-1E cells were treated or not, as described above. Phosphorylation and total protein amount were analyzed by Western blotting as described above. Quantification of three to five experiments is shown. Data are expressed as ratio of phosphorylated on total protein amount and as fold of phosphorylation over basal in cells without treatment. Data are presented as the means±SEM. *P<0.05, **P<0.01, and ***P<0.001 vs stimulus effect in control cells.

FIG. 4: Tpl2 expression in chronic cytokine-treated INS-1 cells and in islets from animal model of type 2 diabetes. A and B: INS-1E β-cells were stimulated or not with IL-1β alone (20 ng/ml) or a cytokine mix (IL-1β (0.2 ng/ml), TNF-α (50 ng/ml), and IFN-γ (30 ng/ml) in RPMI medium for indicated times. Lysates were subjected to Western blotting with antibodies against Tpl2 (1:250), cleaved caspase-3 (1:1000), total and cleaved PARP (1:1000) or β-actin (1:5000). Dotted line represents 100% of protein amount from untreated control cells. Quantification of four independent experiments is shown. C: Proteins from lysates were prepared from Wistar or GK rat islets and subjected to western blotting using Tpl2 antibody (1:250) or β-actin (1:5000). Quantification of six rats for each group is shown. D: Human islets were stimulated or not with a cytokine mix (IL-1β (1000 U/ml), TNF-α (1000 U/ml) and IFN-γ (1000 U/ml)) in RPMI medium containing 0.2% of human albumin for 72 h. Lysates were subjected to Western blotting with antibodies against Tpl2 (1:250) or β-actin (1:5000). Quantification of four independent replicates is shown (each replicate correspond to islets from one human donor). For the entire figure, data are expressed as ratio of different proteins on β-actin protein amount and as the percentage of protein amount in untreated cells (A, B), Wistar rat islets (C), or untreated human islets (D). Data are expressed as the means±SEM. *P<0.05, **P<0.01, and ***P<0.001 vs untreated cells (A and B), Wistar rat islets (C), or untreated human islets (D).

FIG. 5: Apoptotic effects of inflammatory cytokines in INS-1E cells and in mouse pancreatic islets under Tpl2 inhibition. A and C: INS-1E β-cells were treated in RPMI medium containing BSA 0.5% (A) or 7.5% SVF (C) with or without Tpl2 inhibitor (3 μM) during 2 h and then stimulated or not with Tpl2 inhibitor (3 μM) and IL-1β alone (20 ng/ml) for 48 h (A) or the cytokine mixture (IL-1β (0.2 μg/ml), TNFα (50 ng/ml), and IFNγ (30 ng/ml) for 24 h (C). Lysates were subjected to Western blotting with antibodies against cleaved caspase-3 (1:1000), total and cleaved PARP (1:1000) or β-actin (1:5000). Quantification of four to ten independent experiments is shown. Data are expressed as ratio of cleaved caspase-3 or cleaved PARP on β-actin protein amounts, and as fold of these proteins over basal in cells without treatment. Data are presented as the means±SEM. *P<0.05, **P<0.01, and ***P<0.001 vs stimulus effect in control cells. B: INS-1E β-cells were stimulated or not in RPMI medium containing SVF 7.5% with each cytokine alone or a mixture of the three (IL-1β (0.2 ng/ml), TNFα (50 ng/ml), or IFNγ(30 ng/ml) for 24 h. Lysates were subjected to Western blotting with antibodies against cleaved caspase-3 (1:1000) or β-actin (1:5000). Dotted line represents 100% of caspase-3 amount from untreated control cells. Quantification of four independent experiments is shown. Data are expressed as a percentage of cleaved caspase-3 protein amount in untreated cells (caspase-3/β-actin protein amount ratio). Data are presented as the means±SEM. *P<0.05, **P<0.01, and ***P<0.001 vs untreated control cells. D: Isolated mouse islets were stimulated or not with a cytokine mix (IL-1β (1000 U/ml), TNF-α (1000 U/ml) and IFN-γ (1000 U/ml)) in RPMI medium for 24 h. The caspase-3/7 activity was measured using the “Caspase-Glo® 3/7 Assay”. Each column represents the mean±SEM of 8 replicates (each replicate corresponds to one mouse). *P<0.05, **P<0.01, and ***P<0.001 vs stimulus effect in control islets.

FIG. 6: Tpl2 inhibition reduces apoptotic effects of physiological cytokines and chemokines secreted by inflammatory cytokines. RAW264.7 macrophages were maintained in DMEM containing 5% (vol/vol) heat-inactivated fetal bovine serum and antibiotics at 37° C. and 5% CO2/95% air atmosphere. RAW264.7 macrophages were incubated for 24 h with LPS (Lipopolysaccharide) (0.5 ng/ml), and the resulting conditioned medium was transferred onto INS-1E cells treated with or without Tpl2 inhibitor (5 μM). Lysates were subjected to Western blotting with antibodies against cleaved caspase-3 (1:1000), or HSP90 (1:1000). Representative immunoblots and quantification of four experiments are shown. Data are expressed as ratio of cleaved caspase-3 on HSP90 protein amount, and as fold of cleaved caspase-3 over basal in cells in control culture medium without Tpl2 inhibitor treatment. Data are presented as the means±SEM. *P<0.05, vs stimulus effect in control cells.

FIG. 7: Tpl2 inhibition decreases the activation of ERK1/2 induced by cytokines in human pancreatic islets. Human islets were isolated, cultured 24-72 h for recovery in CMRL medium containing 10% SVF, treated with KRBH buffer with or without Tpl2 inhibitor (3 μM) during 2 h and then stimulated or not with a cytokine mix (IL-1β (100 U/ml), TNF-α (500 U/ml), and IFNγ(100 U/ml)) for 20 min. Lysates were subjected to western blot analysis with antibodies against phosphorylated ERK1/2 (1:1000) or β-actin (1:2000). Representative immunoblots and quantification of three independent experiments are shown. Data are expressed as ratio of phosphorylated ERK2 on β-actin amount and as a fold increase over basal in islets without treatment. Data are expressed as the means±SEM. *P<0.05, vs stimulus effect in control cells.

FIG. 8: Effect of in vivo inhibition of Tpl2 on initial and body weights. Five-week-old db/db mice were obtained from Janvier Ltd and fed with a standard diet (4% fat) all over the study. All mice had free access to food and fresh water and were kept on a 12 h-day/12 h-night cycle. Body weights were recorded until the day of sacrifice prior to intraperitoneal (ip) administration of glucose, insulin or the daily injection of 2.5 mg/kg Tpl2 inhibitor or of the corresponding vehicle.

FIG. 9: Effect of in vivo inhibition of Tpl2 on fasting glucose and plasma insulin levels. Five-week-old db/db mice were obtained from Janvier Ltd and fed with a standard diet (4% fat) all over the study. All mice had free access to food and fresh water and were kept on a 12 h-day/12 h-night cycle. Fast and blood glucose concentrations were determined with a (Verio Onetouch, Lifescan, Johnson and Johnson Company) glucometer using blood sampled from the tail vein on mice receiving daily injection of 2.5 mg/kg Tpl2 inhibitor or of the corresponding vehicle. Serum insulin levels were quantified by radioimmunoassay (RIA rat insulin kit, Millipore) using blood sampled from the tail vein on the first day of the study or jugular arteries the day of the sacrifice.

FIG. 10: Effect of in vivo inhibition of Tpl2 on glucose tolerance and insulin tolerance. Five-week-old db/db mice were obtained from Janvier Ltd and fed with a standard diet (4% fat) all over the study. All mice had free access to food and fresh water and were kept on a 12 h-day/12 h-night cycle. Mice received daily injection of 2.5 mg/kg Tpl2 inhibitor or of the corresponding vehicle. Glucose tolerance tests were performed by ip administration of 1-2 g/kg glucose after a 16 h overnight fast and blood glucose concentrations were determined with a (Verio Onetouch, Lifescan, Johnson and Johnson Company) glucometer using blood sampled from the tail vein. Insulin tolerance tests were carried out in a similar manner following the ip administration of 0.75 U insulin per kg body weight to non-fasted mice.

FIG. 11: Anti-apoptotic effect of GLP-1 analog (Exendin-4) /Tpl2 inhibitor combination on INS-1E cells. INS-1E β-cells were treated in RPMI medium containing 7.5% SVF with or without Tpl2 inhibitor (3 μM) and/or Exendin-4 (Ex-4) (20 nM) during 2 h and then stimulated or not with Tpl2 inhibitor (3 μM), Exendin-4 (20 nM) and/or a cytokine mix (IL-1β (0.2 ng/ml), TNF-α (50 ng/ml), and IFN-γ (30 ng/ml) for 24 h. Lysates were subjected to Western blotting with antibodies against cleaved caspase-3 (1:1000), total and cleaved PARP (1:1000) or β-actin (1:5000). Representative immunoblots and quantification of ten independent experiments are shown. Data are expressed as ratio of cleaved caspase-3 or cleaved PARP on β-actin protein amount, and as a percentage of this ratio in cytokine treated cells (called “% of control” in figure, dotted line represents 100% of protein amount). Data are presented as the means±SEM. *P<0.05, **P<0.01, and ***P<0.001 vs cytokine effect in control cells.

FIG. 12: Protection of human islets from cytokine-induced insulin secretion failure by GLP-1 analog (Exendin-4) /Tpl2 inhibitor combination. Human islets were treated in RPMI medium containing 0.2% human albumin with or without Tpl2 inhibitor (3 μM) and/or Exendin-4 (20 nM) during 2 h and then stimulated or not with Tpl2 inhibitor (3 μM), Exendin-4 (20 nM) and/or a cytokine mixture (IL-1β (1000 U/ml), TNF-α (1000 U/ml) and IFN-γ (1000 U/ml) for 72 h, and then submitted to a glucose-response test in KRB buffer. The stimulation index is defined as the ratio of stimulated (20 mM glucose) to basal (2.8 mM glucose) insulin secretion. Each column represents the mean±SEM of 5 replicates (each replicate corresponds to islets from one human donor). *P<0.05, **P<0.01, and ***P<0.001 vs cytokine effect in control cells.

FIG. 13: Anti-apoptotic effect of by another GLP-1 analog (Liraglutide)/Tpl2 inhibitor combination on INS-1E cells. INS-1E β-cells were treated in RPMI medium containing 7.5% SVF with or without Tpl2 inhibitor (3 μM) and/or Liraglutide (20 nM) during 2 h and then stimulated or not with Tpl2 inhibitor (3 μM), Liraglutide (20 nM) and/or a cytokine mix (IL-1β (0.2 μg/ml), TNF-α (50 ng/ml), and IFN-γ (30 ng/ml) for 24 h. Lysates were subjected to Western blotting with antibodies against cleaved caspase-3 (1:1000) or β-actin (1:5000). Representative immunoblots and quantification of 3 independent experiments are shown. Data are expressed as ratio of cleaved caspase-3 on β-actin protein amount, and as a percentage of this ratio in cytokine treated cells (called “% of control” in figure, dotted line represents 100% of protein amount). Data are presented as the means±SEM. *P<0.05, ** P<0.01, and ***P<0.001 vs cytokine effect in control cells.

FIG. 14: Anti-apoptotic effect of GLP-1/DPP-4 inhibitor/Tpl2 inhibitor combination on INS-1E cells. INS-1E β-cells were treated in RPMI medium containing 7.5% SVF with or without Tpl2 inhibitor (3 μM) and/or GLP-1 (20 nM), DPP-4 inhibitor (Sitagliptin, 20 nM) during 2 h and then stimulated or not with Tpl2 inhibitor (3 μM), GLP-1 (20 nM), DPP-4 inhibitor (Sitagliptin, 20 nM) and/or a cytokine mix (IL-1β (0.2 ng/ml), TNF-α (50 ng/ml), and IFN-γ (30 ng/ml) for 24 h. Lysates were subjected to Western blotting with antibodies against cleaved caspase-3 (1:1000) or β-actin (1:5000). Representative immunoblots and quantification of 3 independent experiments are shown. Data are expressed as ratio of cleaved caspase-3 on β-actin protein amount, and as a percentage of this ratio in cytokine treated cells (called “% of control” in figure, dotted line represents 100% of protein amount). Data are presented as the means±SEM. *P<0.05, **P<0.01, and ***P<0.001 vs cytokine effect in control cells.

### EXAMPLES

### Example 1

### In Vitro Inhibition of Tpl2 Kinase in Murine and Human β-Cells as Well as in Murine and Human Pancreatic Islets

Material & Methods

Materials and reagents: RPMI (Roswell Park Memorial Institute medium) culture media, fetal calf serum (FCS), human recombinant IL-1β and TNF-α, and human and rat recombinant IFN-γ were purchased from Invitrogen (Life Technologies SAS, France). Murine IL-1β and TNF-α were purchased from PreProtech (Neuilly, France). Tpl2 kinase inhibitor [4-(3-Chloro-4-fluorophenylamino)-6-(pyridine-3-yl-methylamino)-3-cyano-[1,7]napthyridine] was obtained from Calbiochem (La Jolla, Calif.). The GLP-1 analog, Exendin-4, was obtained from Bachem (Budendorf, Switzerland). Nitrocellulose transfer membranes (Protran) and chromatography paper were obtained from Schleicher & Schuell (Dassel, Germany). High performance chemiluminescence films were purchased from Amerham (GE Healthcare limited, Buckinghamshire, UK). Bicinchoninic acid (BCA) and Copper (II) sulfate solutions, collagenase XI, and Histopaque® 1077 were from Sigma (St. Louis, Mo.). Enhanced chemiluminescence reagents were from Santa Cruz Biotechnology and Perkin Elmer.

Antibodies:

Anti-Tpl2 and HRP-linked anti-mouse IgG antibodies were obtained from Santa Cruz Biotechnology (Santa Cruz, Calif., USA). Anti-ERK1/2 (p44 and p42 MAPK) antibody was obtained from Transduction Laboratories (BD Biosciences Pharmingen, San Diego, Calif.), and anti-β-actin antibody was obtained from Sigma (St. Louis, Mo.). Antibodies against cleaved caspase-3, cleaved and total PARP, total p38 MAPK, total SAPK/JNK (p46 and p54 SAPK/JNK), phospho-p90rsk (Thr573), phospho-MSK-1 (Thr581), phospho-ERK1/2 (Thr202/Tyr204), phospho-p38 MAPK (Thr180/Tyr182), phospho-SAPK/JNK (Thr183/Tyr185) and horseradish peroxidase (HRP)-linked anti-rabbit IgG were obtained from Cell Signaling Technology (New England Biolabs, Beverly, Mass.).

Culture of INS-1E Cells:

The rat β-cell line INS-1E was provided by Dr. Maechler (Department of Cell Physiology and Metabolism, Faculty of Medicine, University of Geneva, Geneva, Switzerland). Cells (passages 60-95) were maintained in culture as monolayer at 37° C. in humidified atmosphere with 5% CO₂, using RPMI 1640 medium containing 11.1 mM glucose, and supplemented with 7.5% heat-inactivated FCS, 1 mM sodium pyruvate, 10 mM HEPES, 2 mM glutamine, 50 μM β-mercaptoethanol, 100 U/ml penicillin, and 100 μg/ml streptomycin. Experiments were performed in INS-1E cells at ˜70% confluence in 6-well plates.

Animals:

Male C57BL/6 mice were obtained from Charles River Laboratories (St. Aubin les Elbeuf, France). Diabetic GK (Goto-Kakizaki) rats were housed and obtained from the adaptive and functional biology unity of CNRS (University of Paris-Diderot, Paris, France). Nondiabetic Wistar rats were used as control. All animals were maintained on a 12 h light, 12 h dark cycle and were provided free access to water and standard rodent diet. They were housed and killed according to the rules of the CNRS Animal Care and Use Committee.

Isolation and Culture of Mouse and Rat Pancreatic Islets:

Pancreatic islets were isolated from male 10- to 12-weeks-old C57BL/6 mice following the injection of approximately 2 ml of 1 mg/ml collagenase XI through the bile duct. The pancreases were then removed, incubated under agitation at 37° C. for 9 minutes to complete the digestion and the islets were separated from the exocrine pancreatic tissue using a Histopaque-1077 gradient. The islets were then washed in cold PBS supplemented with 1.2 mM CaCl₂, 1 mM MgCl₂, and 5.6 mM glucose, handpicked under microscope, separated in groups composed of 200-300 islets and maintained in culture at 37° C. (95% air and 5% CO₂) in RPMI 1640 containing 11.1 mM glucose and supplemented with 10% FCS, 2 mM glutamine, 100 units/ml penicillin and 100 μg/ml streptomycin for at least 24 h before being used. Islets from 8-10 weeks-old male Wistar and GK rats were isolated at the adaptive and functional biology unity of CNRS (University of Paris-Diderot, Paris, France).

Isolation and Culture of Human Pancreatic Islets:

Human pancreases were harvested from five brain-dead non-obese non-diabetic donors in agreement with the French Agence de la Biomedecine and the local Institutional Ethical Committee. Human islets were isolated at the Diabetes Cellular Therapy Laboratory (Institute for Research in Biotherapy, Montpellier, France) or at the Cell Isolation and Transplantation Center (University of Geneva, Geneva, Switzerland) according to a slightly modified version of the automated method (Ricordi et al, 1988; Bucher et al, 2005). Islets were cultured in CMRL 1066 (Mediatech, Herndon, Va.) medium containing 5.6 mM of glucose and supplemented with 10% FCS, 100 UI/ml penicillin, 100 mg/ml streptomycin and 2 mM glutamine for recovery during 1 to 5 days before drug exposure.

Tpl2 siRNA in INS-1E Cells:

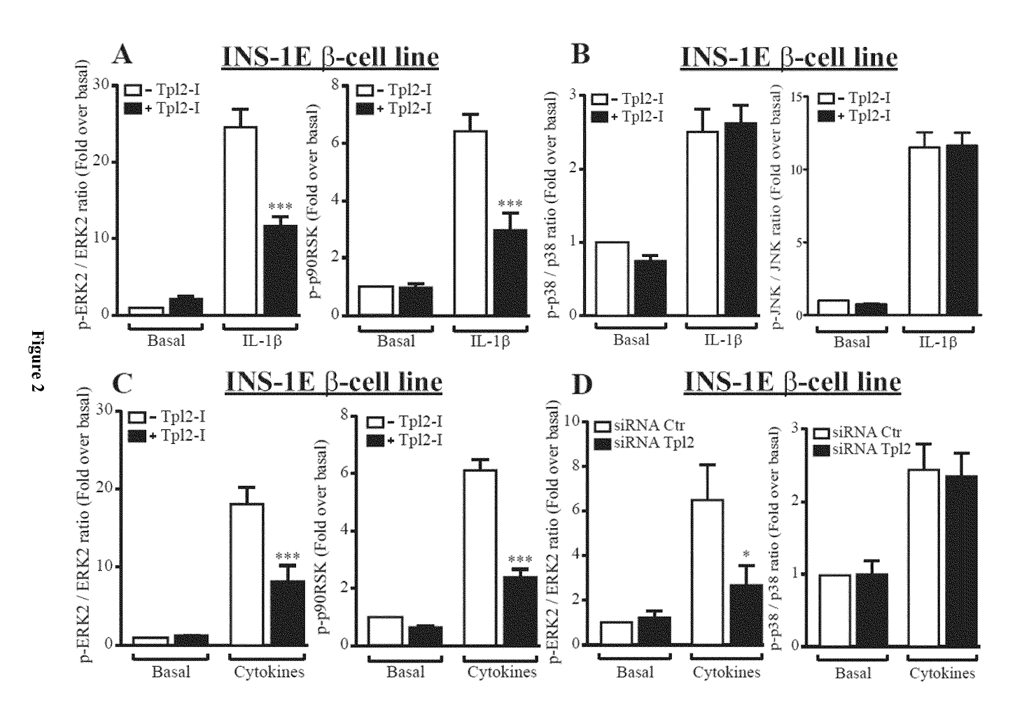

Tpl2 expression was specifically silenced in INS-1E cells using a validated set of 4 different 19-nucleotides siRNA duplexes (“ON-TARGETplus SMARTpool”, L-091828-01-0005) purchased from Dharmacon (ABgene Ltd, part of Thermo Fisher Scientific, Waltham, Mass.). The specificity of our siRNA approach was ascertained using the siRNA control (Positive and negative controls were “ON-TARGETplus cyclophilin B control pool-rat” and “ON-TARGETplus Non-targeting pool-rat” from Dharmacon), which failed to induce any change in the expression of any of proteins studied and used as internal loading control as shown in FIGS. 2D and 2F. Briefly, groups of 500,000 INS-1E cells were maintained in culture in the absence of penicillin and streptomycin for 24 h before being transfected with 40 nM siRNA-Lipofectamine™ 2000 complexes prepared in Opti-MEM medium at a 2:1 ratio. Medium was replaced 6 h after transfection with fresh antibiotic-free RPMI medium supplemented with 7.5% heat-inactivated FCS. A second transfection was performed 24 h after the first one to improve the transfection efficiency. The transfection efficiency was assessed using the “siGLO Green Transfection Indicator”. All assays were performed at least 50 h after the first transfection.

Drug Exposure:

To determine the implication of Tpl2 in effects of an acute cytokine stimulation on kinase phosphorylation, short term experiments were performed in Krebs-Ringer Bicarbonate (KRB) buffer: glucose-free HEPES-balanced KRB (KRBH) for INS-1E cells (135 mM NaCl, 3.6 mM KCl, 0.5 mM NaH₂PO₄, 0.5 mM MgCl₂, 1.5 mM CaCl₂, 5 mM NaHCO₃, and 10 mM HEPES, pH 7.4, containing 0.1% BSA); and KRB buffer for mouse islets (120 mM NaCl, 4.7 mM KCl, 1.2 mM KH₂PO₄, 1.2 mM MgSO₄, 2.5 mM CaCl₂, and 24 mM NaHCO₃, pH 7.4, containing 0.1% BSA and 1.1 mM glucose). INS-1E cells or mouse islets (200-300 islets per condition) were preincubated at 37° C. during 2 h with or without Tpl2 inhibitor (3 μM) and then incubated for different times (0, 5, 10, 20, or 30 min) with or without Tpl2-inhibitor (3 μM), glucose (10 mM), IL-10 alone (10000 U/ml, 20 ng/ml), a cytokine mix (IL-1β (100 U/ml, 0.2 ng/ml), TNFα (500 U/ml, 50 ng/ml), and IFNγ (100 U/ml, 30 ng/ml), or Exendin-4 (20 nM).

To evaluate the role of Tpl2 in conditions of chronic exposure to cytokines, long term experiments were performed in RPMI 1640 medium containing 2 mM glutamine, 100 U/ml penicillin, 100 μg/ml streptomycin, and glucose (11.1 mM for INS-1E cells and mouse islets, and 5.6 mM for human islets). The medium was supplemented by heat-inactivated FCS (7.5% for INS-1E cells and 10% for mouse islets) or by albumin (0.5% of BSA for IL-1β alone on INS-1E cells, and 2% of human albumin for human islets). For INS-1E cells, the medium contains 1 mM sodium pyruvate, 10 mM HEPES, and 50 μM β-mercaptoethanol. INS-1E cells, mouse (5-10 islets per condition) or human islets (500-2000 IEQ per condition) were preincubated at 37° C. during 2 h with or without Tpl2 inhibitor (3 μM) and Exendin-4 (20 nM), and then incubated for different times (0, 8, 16, 24, 36, 48 or 72 h) with or without Tpl2-inhibitor (3 μM), IL-1β alone (10000 U/ml, 20 μg/ml), a cytokine mix (IL-1β (100 U/ml, 0.2 ng/ml), TNF-α (500 U/ml, 50 ng/ml), and IFN-γ (100 U/ml, 30 ng/ml) for INS-1E cells and mouse islets; and IL-1β (1000 U/ml, 2 ng/ml), TNF-α (1000 U/ml, 28 ng/ml) and IFN-γ (1000 U/ml, 833 ng/ml) for human islets), and/or Exendin-4 (20 nM). All experiments on human islets were performed at Diabetes Cellular Therapy Laboratory of Montpellier (Institute for Research in Biotherapy, Montpellier).

Western Blotting:

Following experiments, INS-1E cells, mouse (200-300 islets per condition) or rat (300-500 islets per condition) islets were washed once with cold PBS and lysed in a cold lysis buffer (50 mM HEPES, 1 mM EDTA, 1 mM phenylmethylsulfonyl fluoride (PMSF), 1 mM Na₃VO₄, 10 mM pyrophosphate, 100 mM NaF, 1% Triton X-100, 0.1% SDS, and 1 mg/ml bacitracin for INS-1E cells; 50 mM HEPES, 4 mM EDTA, 1 mM PMSF, 1 mM Na₃VO₄, 10 mM pyrophosphate, 100 mM NaF, 1% Nonidet P-40, 1 mg/ml leupeptin, 1 mg/ml aprotinin for mouse and human islets). For a better lysis, islets were frozen in liquid azote before adding lysis buffer and sonicated. Cell or islet lysates were then clarified by centrifugation (13,000 rpm for 30 min at 4° C.), and protein concentration was determined using the BCA method. Protein were denaturated by boiling 5 min in a Laemmli sample buffer, and equal amounts of proteins (15-30 μg of protein/lane) were separated through a 10 or 12.5% SDS-PAGE and transferred to nitrocellulose membranes. After blocking 1 h at room temperature (RT), membranes were incubated overnight at 4° C. with specific primary antibodies (1:250 to 1:4000 dilutions), and then incubated 1 h at RT with horseradish peroxidase-linked secondary antibody (1:2000). Proteins were visualized by enhanced chemiluminescence detection. Autoradiographs were digitized, and the band density was analyzed using ImageJ software (National Institutes of Health, Bethesda, Md.).

Cell and Pancreatic Islet Viability:

The presence of apoptotic INS-1E cells was investigated by evaluating by Western blotting the emergence of the 17 kDa cleaved form of caspase-3 which corresponds to the active pro-apoptotic form of caspase-3, and the 89 kDa cleaved form of the nuclear enzyme poly(ADP-ribose) polymerase (PARP) involved in DNA repair and cell survival. Mouse islet apoptosis was assessed by measurement of caspase-3/7 activity using the “Caspase-Glo® 3/7 Assay” (Promega Corp. Madison, Wis., USA) according to the manufacturer's instructions. This kit is based on the cleavage of the DEVD sequence of a luminogenic substrate by the caspases 3 and 7 resulting in a luminescent signal. Briefly, caspase-3/7-Glo reagent was added after the 24 h incubation of islets in 96-well plates (10 islets of approximately equivalent size per well), and the samples were incubated at 37° C. for 2 h. Luminescence was measured using a TECAN infinite 200 plate reader.

Insulin Secretion Assays:

After a 72 h incubation period in RPMI with or without Tpl2 inhibitor and cytokines, human isolated islets (3×50 human islet equivalent (IEQ) per condition) were preincubated for 1 h at 37° C. for stabilization in KRB buffer (24 mM NaHCO₃, 120 mM NaCl, 4.7 mM KCl, 1.2 mM KH₂PO₄, 1.2 mM MgSO₄, 2.5 mM CaCl₂, and 10 mM HEPES, pH7.4, BSA 0.1%) containing glucose 2.8 mM, followed by a 1 h incubation at 2.8 mM and an additional 1 h at glucose 20 mM. Aliquots from the incubation buffers were collected, cleared by centrifugation, and frozen. Extraction of total islet insulin content was performed by ethanol acid followed by sonications. Insulin release and content were measured by radioimmunoassay (Milipore, SAS FRANCE) following manufacturer's instructions, and the stimulation index was defined as the ratio of stimulated to basal insulin secretion (ng/ml/islet/hour) and corrected by the insulin content (ng/ml/islet).

Statistical Analysis:

All the experiments were independently repeated at least three times. Results were expressed as the means±SEM for N independent experiments. The statistical significance between means was assessed by unpaired Student t-test, or by ANOVA followed by Newman-Keuls or Bonferroni post-hoc analyses. Analyses were performed with GraphPad Prism 5 software. A P value <0.05 was considered significant. *, P<0.05, **, P<0.01, ***, P<0.001.

Inhibition of Tpl2 Reduces Apoptotic Effects of Physiological Cytokines and Chemokines Secreted by Inflammatory Macrophages:

RAW264.7 macrophages were maintained in DMEM containing 5% (vol/vol) heat-inactivated fetal bovine serum and antibiotics at 37° C. and 5% CO2/95% air atmosphere. RAW264.7 macrophages were incubated for 24 h with LPS (Lipopolysaccharide) (0.5 ng/ml), and the resulting conditioned medium was transferred onto INS-1E cells treated with or without Tpl2 inhibitor (5 μM). Lysates were subjected to Western blotting with antibodies against cleaved caspase-3 (1:1000), or HSP90 (1:1000). Representative immunoblots and quantification of four experiments are shown. Data are expressed as ratio of cleaved caspase-3 on HSP90 protein amount, and as fold of cleaved caspase-3 over basal in cells in control culture medium without Tpl2 inhibitor treatment. Data are presented as the means±SEM. *P<0.05, vs stimulus effect in control cells.

Tpl2 Inhibition Decreases the Activation of ERK1/2 Induced by Cytokines in Human Pancreatic Islets:

Human islets were isolated, cultured 24-72 h for recovery in CMRL medium containing 10% SVF, treated with KRBH buffer with or without Tpl2 inhibitor (3 μM) during 2 h and then stimulated or not with a cytokine mix (IL-1β (100 U/ml), TNF-α (500 U/ml), and IFNγ (100 U/ml)) for 20 min. Lysates were subjected to western blot analysis with antibodies against phosphorylated ERK1/2 (1:1000) or b-actin (1:2000). Representative immunoblots and quantification of three independent experiments are shown. Data are expressed as ratio of phosphorylated ERK2 on b-actin amount and as a fold increase over basal in islets without treatment. Data are expressed as the means±SEM. *P<0.05, vs stimulus effect in control cells.

Results

Tpl2 Expression and Activation.

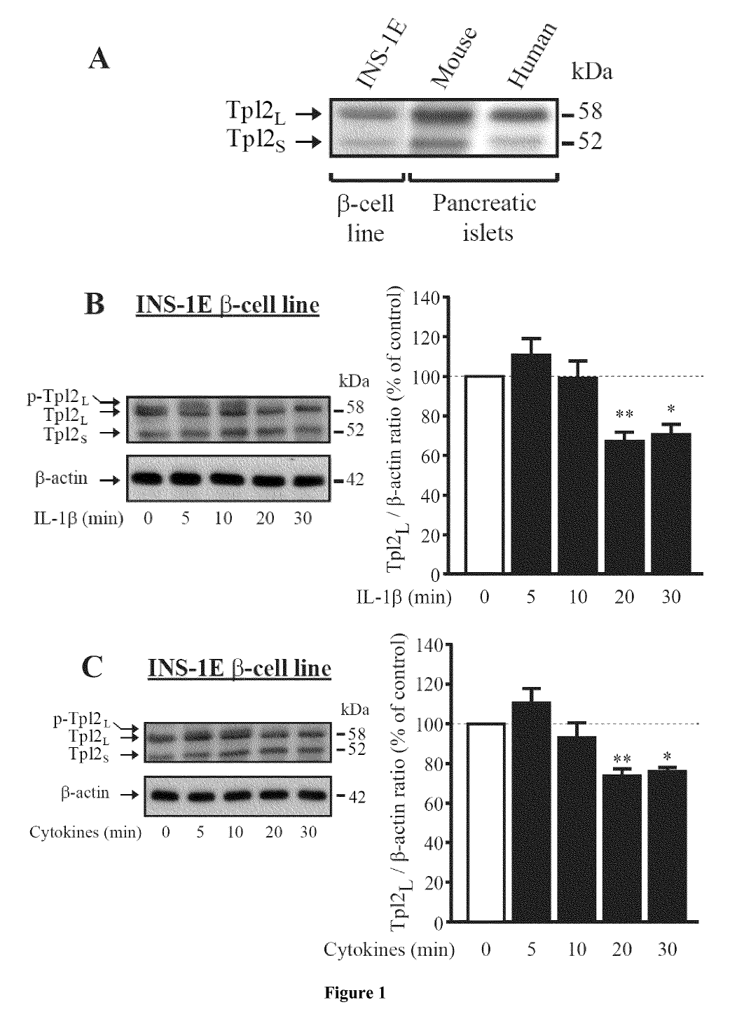

An anti-Tpl2 antibody detected two bands of 58 and 52 kDa in INS-1E cells, mouse and human pancreatic islets (FIG. 1A) which correspond to the long (Tpl2_{L}) and the short (Tpl2_{S}) isoforms of Tpl2 that have been described to arise from an alternative translational initiation (Aoki et al, 1993). Tpl2_{L} was found to be preferentially expressed in INS-1E cells, mouse and human pancreatic islets in comparison to Tpl2_{S} (FIG. 1A). The inventors investigated whether Tpl2 was activated by IL-1β or by a mixture of three cytokines (IL-1β, TNF-α, IFN-γ), by evaluating its degradation which has been shown to be tightly coupled to its activation (Vougioukalaki et al, 2011; Gantke et al, 2011). Western blotting analysis revealed that IL-1β and cytokine mixture stimulation induced a Tpl2_{L} band shift likely indicative of a Tpl2_{L} phosphorylation (seen at 5-10 min of stimulation), and significantly decreased total Tpl2 protein expression after 20 min of treatment and for at least 30 min (FIGS. 1B and 1C). Notably, Tpl2_{L} was preferentially phosphorylated and degraded (FIGS. 1B and 1C).

Role of Tpl2 in ERK1/2 Activation.

To pursue the study of Tpl2 biology in β-cells, a tool compound was needed, and, among the designed Tpl2 inhibitors, the inventors used the Tpl2 inhibitor from Calbiochem (Web site: http://www.millipore.com/catalogue/item/616373-1 mg). This Tpl2 inhibitor was used at 3 μM concentration since concentrations below 5 μM are known to display significant selectivity over other related kinases such as MEK, MK2, Src, protein kinase C, and EGF receptor. No other protein kinase has been found to be inhibited or activated by Tpl2 inhibitor at a concentration (<5 μM) that prevents activation of the ERK1/2 cascade. As shown in FIG. 2A, when used at 3 μM, Tpl2 inhibitor suppressed 60% of IL-1β-induced ERK1/2 phosphorylation in INS-1E cells, indicating that IL-1(3-stimulated phosphorylation of ERK1/2 requires Tpl2 activation. The p90 ribosomal S6 kinase (p90RSK), known to be located downstream of ERK1/2, was inhibited to the same extent (FIG. 2A). The specificity of the Tpl2 inhibitor treatment was ascertained by the fact that concentration of 3 μM, and even high concentration of 10 μM (data not shown), had no effect on IL-1β-induced p46/p54 c-Jun N-terminal kinases (JNK) and p38 phosphorylation (FIG. 2B). The 50-60% suppression of cytokine-induced ERK1/2 phosphorylation by the Tpl2 inhibitor treatment further demonstrates that cytokine-stimulated phosphorylation of ERK1/2 also requires Tpl2 activation (FIG. 2C). The phosphorylation of p90RSK induced by the cytokines was inhibited to the same extent (FIG. 2C).

They performed a siRNA knockdown strategy to silence the expression of Tpl2 protein in INS-1E cells. As seen in FIG. 2D, 50% decrease of Tpl2 (Tpl2_{L} and Tpl2ₛ) protein expression inhibited by ˜50% the ERK1/2 phosphorylation induced by the cytokines in Tpl2 siRNA-transfected INS-1E cells compared with control siRNA-transfected INS-1E cells. Phosphorylation of p38 remained unaffected in these conditions (FIG. 2D). Using pharmacological inhibition or siRNA invalidation, these results indicate that the predominant pathway that is activated by acute IL-1β and cytokine stimulation in INS-1E cells through basal cellular expression of Tpl2 is the one leading to ERK1/2.

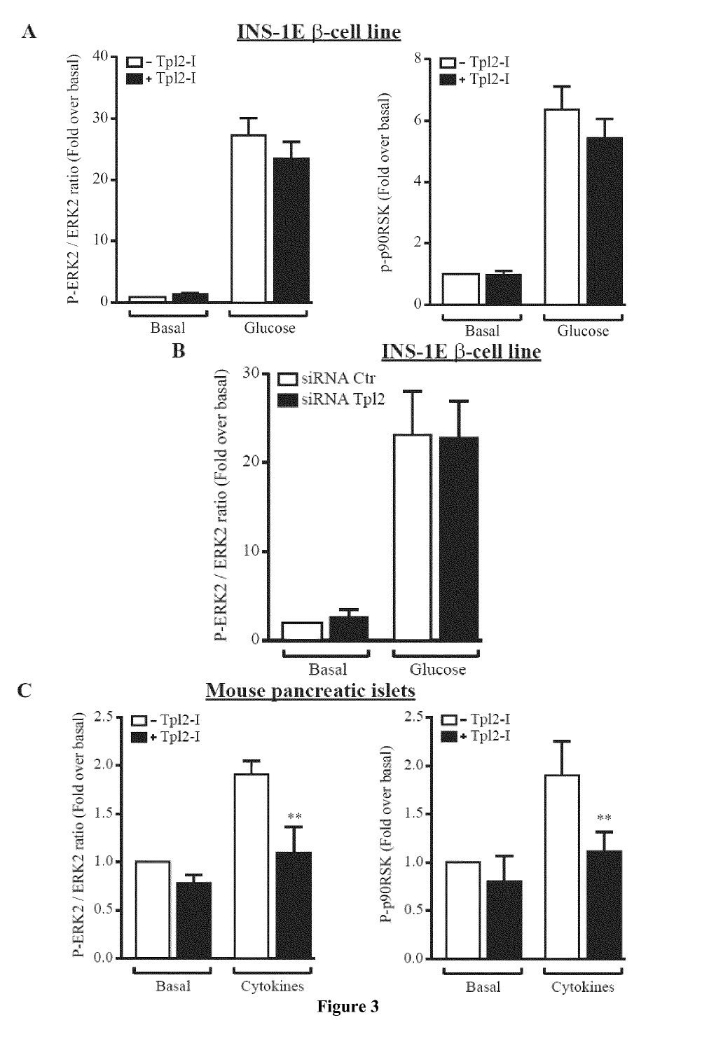

Importantly, neither glucose-induced ERK1/2 nor p90RSK phosphorylations, described to play a key role in glucose-mediated β-cell survival (Costes et al, 2006), were modified neither by Tpl2 inhibitor treatment (FIG. 3A) nor Tpl2 siRNA transfection of INS-1E cells (FIG. 3B). Together, these data demonstrate a Tpl2 involvement in ERK1/2 activation specifically in response to inflammatory stimulus. Moreover, glucose did not decrease total Tpl2 protein expression (data not shown), compared to stimulation by IL-1β or cytokines (FIGS. 1B and 1C), suggesting that glucose does not activate Tpl2 in β-cells.

To confirm in a more physiological model, the involvement of Tpl2 in the cytokine-induced ERK1/2 phosphorylation evidenced in INS-1E cells, they used islets of Langerhans isolated from C57BL/6 mice. Treatment of pancreatic islets with cytokines stimulated phosphorylation of ERK1/2 and p90RSK (FIG. 3C). A Tpl2 inhibitor treatment was efficient at inhibiting ERK1/2 and p90RSK phosphorylation (FIG. 3C).

Tpl2 Expression is Increased by Chronic Cytokine Treatment and in Islets from Animal Model of Type 2 Diabetes.

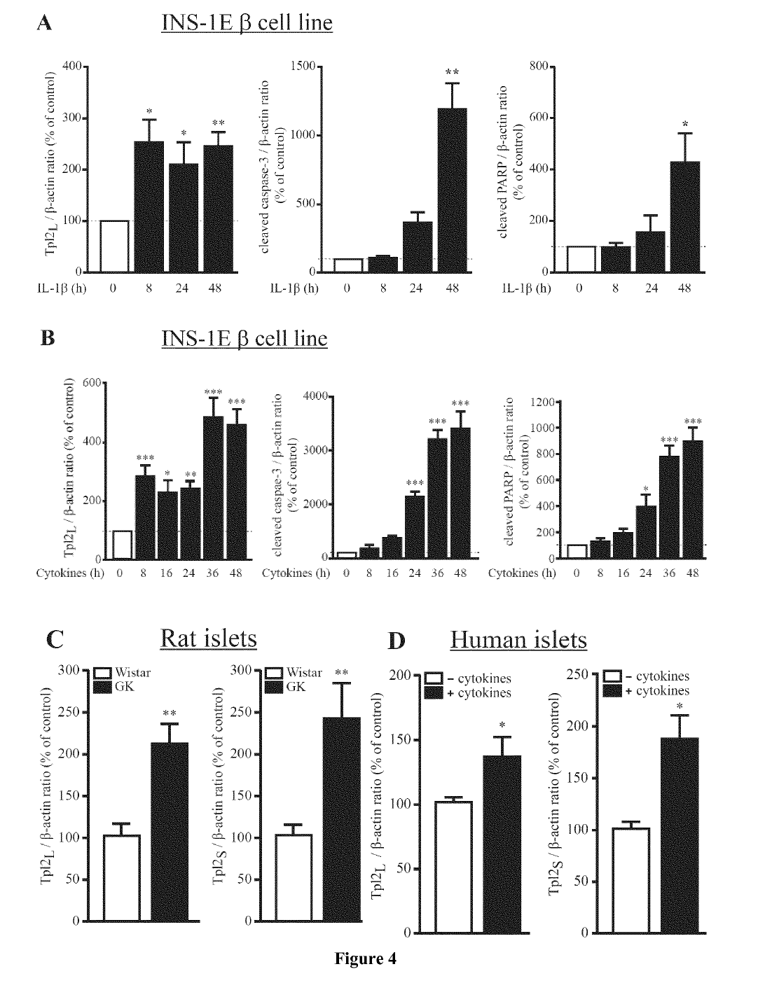

While acute treatment (20-30 min) with IL-1β or a mixture of cytokines induced Tpl2 degradation (FIGS. 1B and 1C), prolonged exposure to IL-1β or cytokines (8 to 24 h) increased both Tpl2_{L} and Tpl2_{S} protein expression by ˜2 to 3 fold in INS-1E cells (FIGS. 4A and 4B). As control, β-actin protein levels remained unaffected (Data not shown). Tpl2_{L} and Tpl2_{S} levels markedly increased with IL-1β or cytokines before the emergence of detectable cleaved caspase-3 and cleaved PARP (FIGS. 4A and 4B). Long term treatment by the cytokines further increased Tpl2_{L} and Tpl2_{S} protein expression by ˜5 fold between 24 to 48 h (FIG. 4B).

The inventors next determined whether diabetogenic environment (hyperglycaemia and inflammation) leads to Tpl2 protein upregulation in vivo. They used the GK rat model, one of the best characterized animal models of spontaneous type 2 diabetes (Portha et al, 2009). GK rats display hyperglycaemia, and GK rat islets feature increased expression of several inflammatory markers including IL-1β and macrophage infiltration (Ehses et al, 2009). As seen in FIG. 4C, significant ˜2 and 2.5 fold increases in Tpl2_{L} and Tpl2_{S} protein expression respectively were found in pancreatic islets isolated from GK rats compared to control normal Wistar rats, demonstrating that diabetogenic environment leads to up-regulation of Tpl2 proteins in vivo.

Level of Tpl2 protein expression was also evaluated in human pancreatic islets. As seen in FIG. 4D, a chronic cytokine treatment (72 h) significantly increased by 1.5 and 2 fold Tpl2_{L} and Tpl2_{S} protein expression in human islets.

Inhibition of Tpl2 Reduces Apoptotic Effects of Inflammatory Cytokines in INS-1E Cells and Mouse Pancreatic Islets.

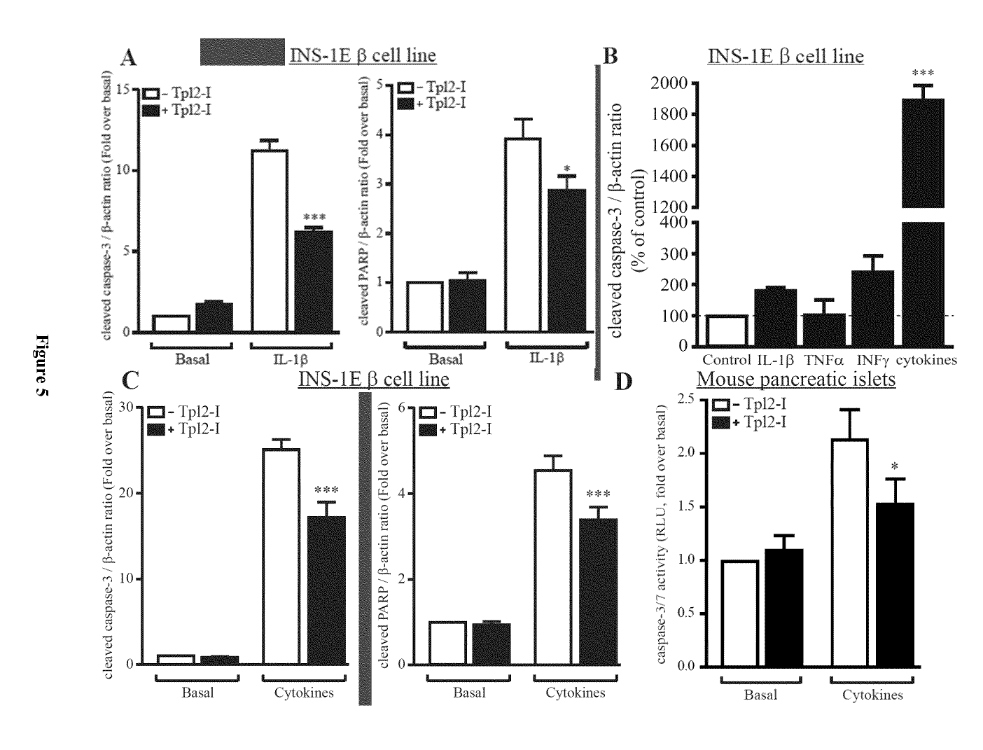

They next determined whether Tpl2 inhibition might modify the deleterious pro-apoptotic effects of IL-1β or cytokines by measuring levels of cleaved forms of caspase-3 and PARP, key executioners and markers of apoptosis. As seen in FIGS. 5A and 5C, long term treatment of INS-1E cells with Tpl2 inhibitor alone did not increase INS-1E cell apoptosis. Exposure of INS-1E cells to IL-1β for time period >24 h led to significant apoptosis (FIGS. 4A and 5A). Notably, decreases in both cleaved caspase-3 and cleaved PARP levels induced by IL-1β were observed (˜45 and ˜30%, respectively) in INS-1E cells treated with Tpl2 inhibitor (FIG. 5A). Fully consistent with previous published observations, level of cleaved caspase-3 was drastically increased by the mixture of the three cytokines compared to the levels induced by the separated cytokine (FIG. 5B). They further verified whether Tpl2 inhibitor treatment may also be efficient at decreasing this drastic apoptotic level. Interestingly, decreases in both cleaved caspase-3 and cleaved PARP levels induced by the mixture of cytokines were observed in INS-1E cells treated with Tpl2 inhibitor (˜30 and ˜25%, respectively) (FIG. 5C).

The potential involvement of Tpl2 in the cytokine-induced apoptosis was further investigated in isolated mouse islets. Notably, treatment of pancreatic islets with Tpl2 inhibitor decreased by ˜50% the level of cleaved caspase-3/7 activity induced by the mixture of cytokines (FIG. 5D).

Inhibition of Tpl2 Reduces Apoptotic Effects of Physiological Cytokines and Chemokines Secreted by Inflammatory Macrophages.

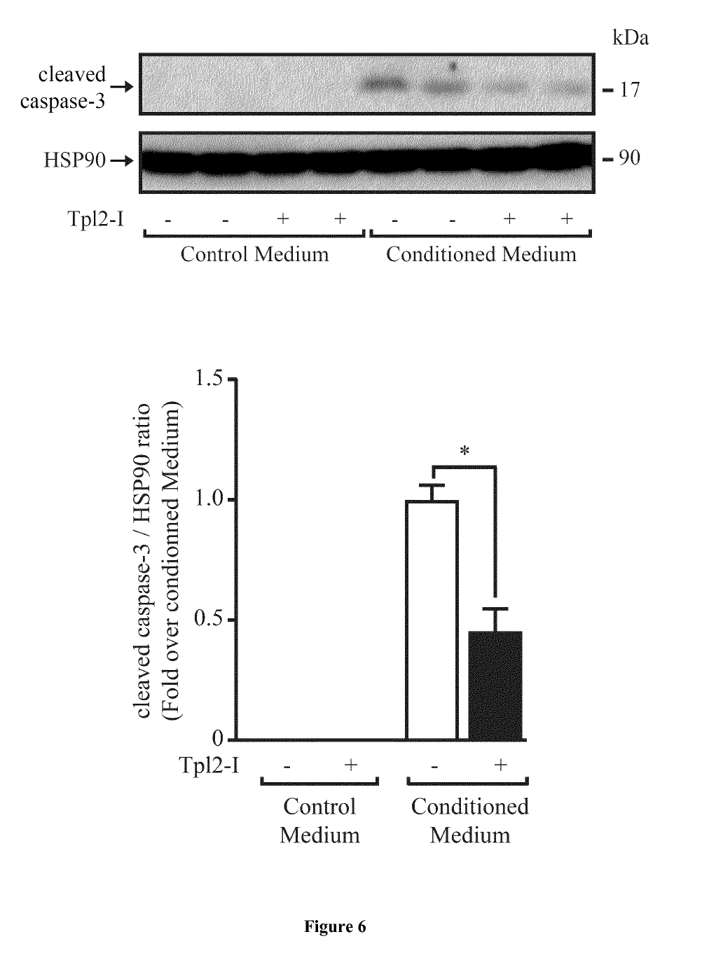

To determine whether Tpl2 inhibition could protect pancreatic β-cell against apoptosis induced a more physiological cocktail of cytokines, the macrophage lineage RAW264.7 was activated by LPS (Lipopolysaccharide). The INS-1E cells were cultured in the presence of this conditioned medium containing several cytokines and chemokines secreted by activated macrophages, like IL-1β, TNF-α and IL-6. Pharmacological inhibition of Tpl2 decreases by around 55% the level of cleaved caspase-3 induced by 24 h of culture of INS-1E cells in this conditioned medium (FIG. 6).

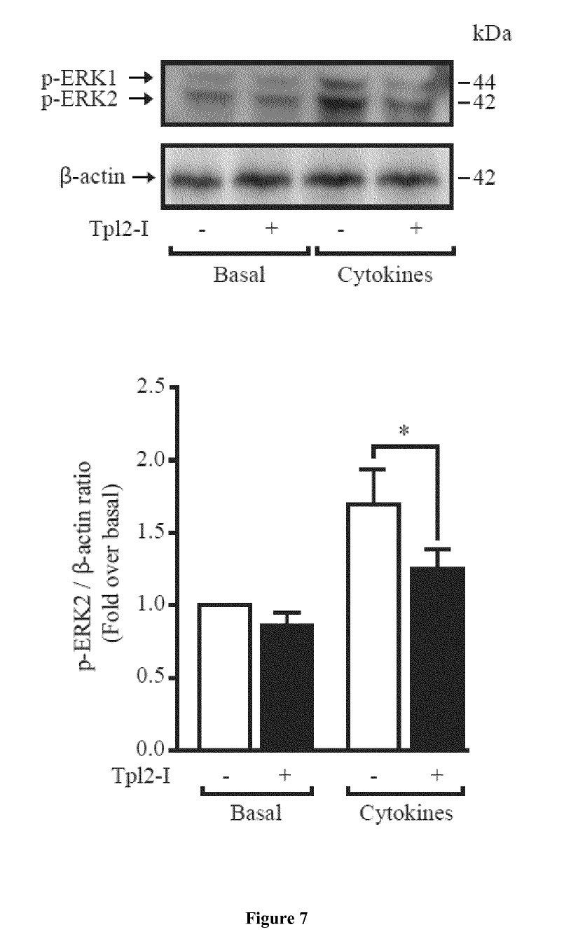

Tpl2 inhibition decreases the activation of ERK1/2 induced by cytokines in human pancreatic islets: We verified that a treatment of human islet with the Tpl2 inhibitor was significantly efficient at inhibiting the phosphorylation/activation of ERK1/2 induced by the cytokine mixture. These results indicate that cytokine-stimulated phosphorylation of ERK1/2 signaling pathway in human pancreatic islets requires Tpl2 activation (FIG. 7).

### Example 2

### In Vivo Inhibition of Tpl2 Kinase Slows the Progression of Type 2 Diabetes in Db/Db Mice

Improvement of Glucose homeostasis in prediabetic and diabetic db/db mice with significant reduction in fasting plasma glucose, fasting insulinemia and improvement of insulin sensitivity.

Material & Methods

Five-week-old db/db mice were obtained from Janvier Ltd and fed with a standard diet (4% fat) all over the study. All mice had free access to food and fresh water and were kept on a 12 h-day/12 h-night cycle. Body weights were recorded until the day of sacrifice prior to intraperitoneal (ip) administration of glucose, insulin or the daily injection of 2.5 mg/kg Tpl2 inhibitor or of the corresponding vehicle. Glucose tolerance tests were performed by ip administration of 1-2 g/kg glucose after a 16 h overnight fast and blood glucose concentrations were determined with a (Verio Onetouch, Lifescan, Johnson and Johnson Company) glucometer using blood sampled from the tail vein. Insulin tolerance tests were carried out in a similar manner following the ip administration of 0.75 U insulin per kg body weight to non-fasted mice. Serum insulin levels were quantified by radioimmunoassay (RIA rat insulin kit, Millipore) using blood sampled from the tail vein on the first day of the study or jugular arteries the day of the sacrifice.

Results

The progressive development of type 2 diabetes as observed in db/db mice is associated with increased body weight, insulin resistance, hyperglycemia and impaired glucose tolerance. Therefore, to determine whether Tpl2 inhibition has the potential to prevent and/or treat type 2 diabetes we monitored these physiological parameters over a 2-week-study using 6-week-old db/+ and db/db mice divided in 3 groups that were treated either with the daily ip injection of 2.5 mg/kg of the Tpl2 inhibitor or of the corresponding vehicle.

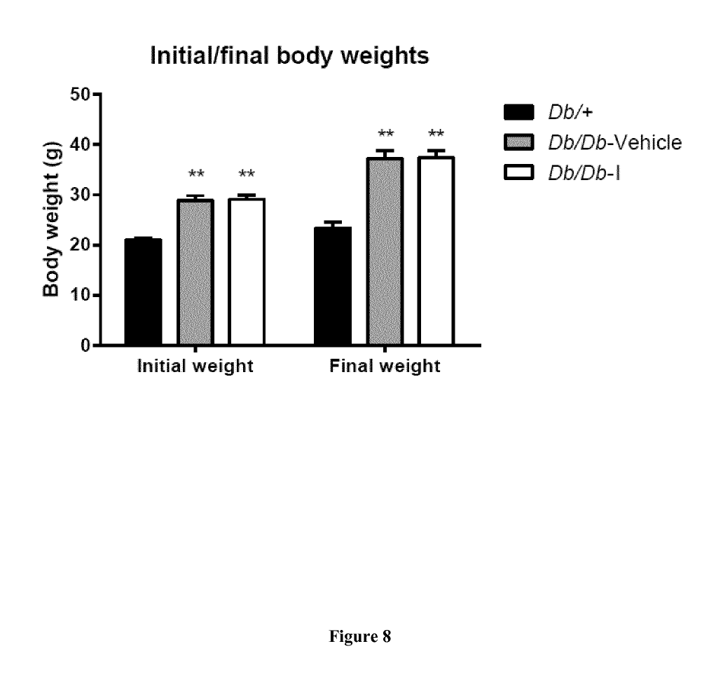

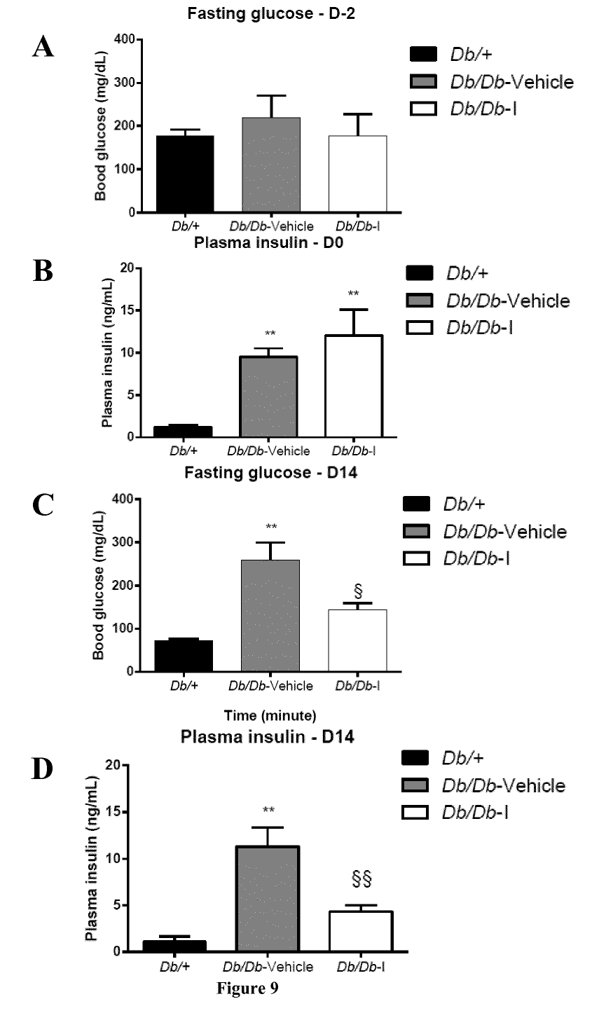

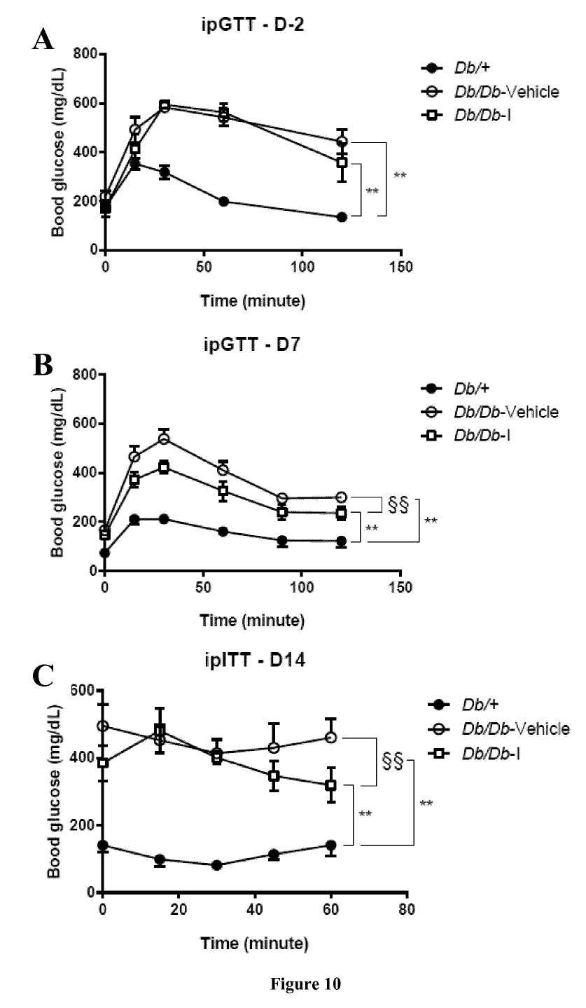

The initial body weights and fasting insulin levels were significantly higher in 6-week-old db/db mice as compared with db/+ animals (FIGS. 8 and 9). In contrast, fasting blood glucose concentrations were not statistically different between groups suggesting that the 6-week-old db/db mice had developed hyperinsulinemia but not diabetes before the start of the study. As a consequence, the tolerance to 2 g/kg glucose challenges was impaired in 6-week-old db/db mice compared to db/+ animals 2 days before the first ip injections (FIG. 10). Importantly, body weights, glucose tolerance, fasting glucose and insulin levels were similar between the 2 groups of db/db mice.

Interestingly, the tolerance to glucose challenges was significantly improved in Tpl2 kinase inhibitor-injected db/db mice already after 7 days of treatments (FIG. 10B). This improvement to glucose tolerance was also associated with a significant reduction fasting blood glucose and serum insulin levels as observed on day 14 (FIG. 9). Thus, the blood glucose concentrations and serum insulin levels at fast were respectively reduced from 258.6±40.7 mg/dL and 11.3±1.2 ng/ml to 144.0±15.5 mg/dL and 4.3±0.4 ng/ml in db/db mice treated with the Tpl2 kinase inhibitor (FIG. 9). Moreover, whereas vehicule-administered db/db mice showed significantly lower insulin-dependent reductions in blood glucose as assessed by insulin tolerance tests, consistent with the development of insulin resistance in db/db animals. We observed a significant improvement in insulin sensitivity after 14 days of treatment with the Tpl2 kinase inhibitor (FIG. 10 C). Finally, the total body weights were significantly increased in all groups after 2 weeks of treatment and these increases were more pronounced in db/db mice compared to db/+ animals (FIG. 8). More importantly, body weights were not different between vehicule- and Tpl2 kinase inhibitor-treated db/db mice suggesting that the daily ip administration of 2.5 mg/kg Tpl2 kinase inhibitor was not toxic during the 2 weeks of investigation (FIG. 8).

Summary of Key Issues from In Vivo Studies

Improvement of glucose homeostasis in prediabetic and diabetic db/db mice with significant reduction in fasting plasma glucose, fasting insulinemia and improvement of insulin sensitivity.
No effect on the body weights
Improvement of glucose tolerance in db/db mice
Improvement of fasting hyperglycemia (This observation allow us to propose that: by lowering blood hyperglycemia Tpl2 inhibition may reduce microvascular and macrovascular complications, cardiovascular risk factors, lessen cancer risk, and improve markers associated with longevity).
Improvement of plasma insulinemia
Improvement of insulin sensitivity

### Example 3

### Combination of Tpl2 Kinase Inhibitor and GPL-1 Agonist (Exendin-4) Useful for Preventing or Treating Diabetes

Material & Methods

Materials & methods have been previously described (especially in the section “Drug exposure”, where combination treatment of Tpl2 inhibitor and exendin-4 is disclosed).

Results

Combination of Tpl2 Inhibitor and GLP-1 Analog Produces Powerful Anti-Apoptotic Effects on INS-1E Cells.

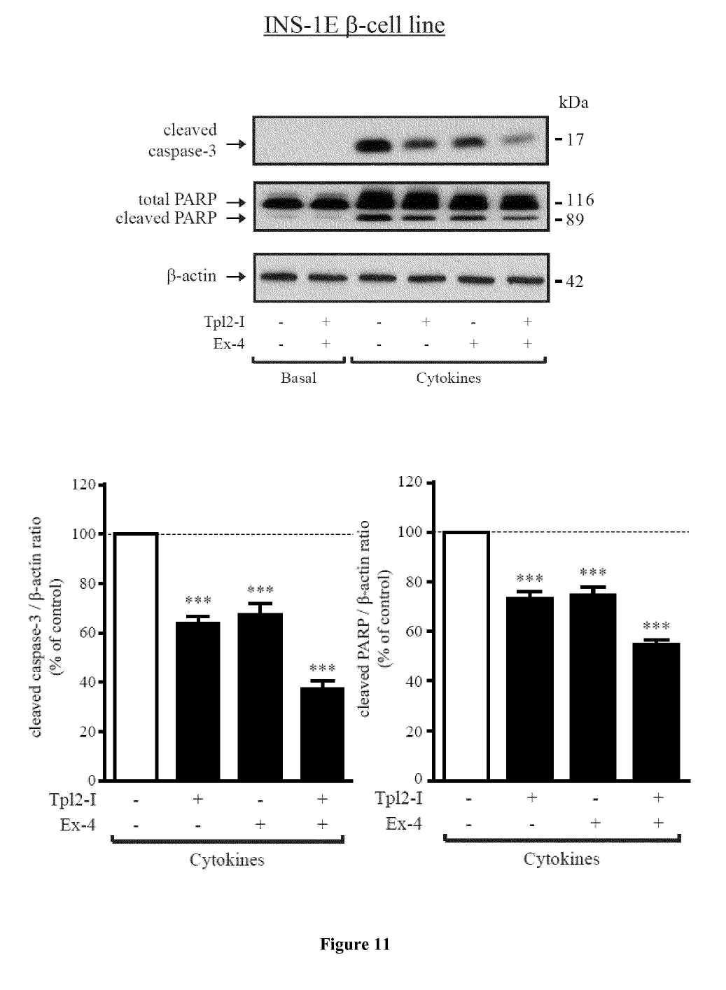

Pre-clinical and clinical studies have shown that GLP-1 receptor agonists (such as Exendin-4) have modest anti-inflammatory effects (Pugazhenthi et al, 2010). In order to verify whether simultaneous inhibition of Tpl2 may improve the GLP-1 agonist beneficial effect on β-cell survival in vitro, the effects of Tpl2 inhibitor alone, Exendin-4 alone or a Tpl2 inhibitor/Exendin-4 combination were first investigated on INS-1E cells submitted to the deleterious effects of cytokines. Importantly, combination of pharmacological inhibition of Tpl2 and Exendin-4 treatment produce more powerful anti-apoptotic effects on INS-1E cells than each compound alone (FIG. 11).

Combination of Tpl2 Inhibitor and GLP-1 Analog Protects Human Pancreatic Islets from Cytokine-Induced Insulin Secretion Failure.

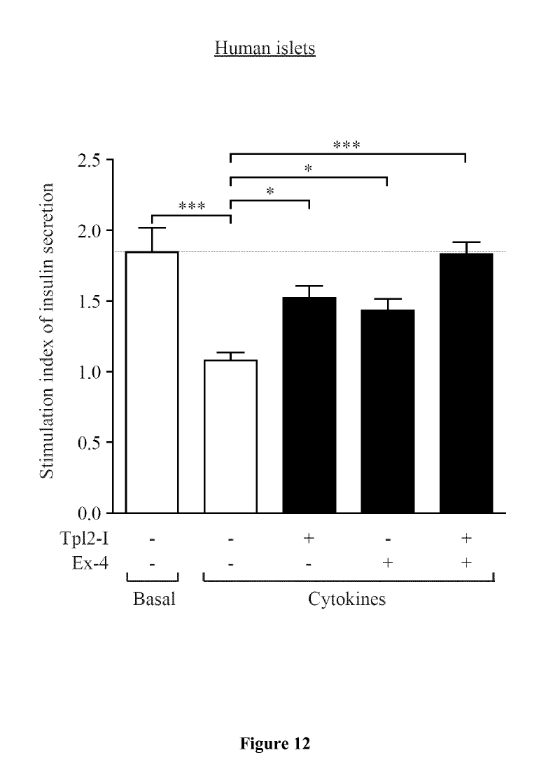

Chronic cytokine exposure of β-cells deteriorates not only β-cell survival but also insulin secretion (Donath et al, 2011). Based on the remarkable anti-inflammatory effects of Tpl2 inactivation observed in INS-1E and mice islet, the inventors ultimately verified whether inactivation of Tpl2 may prevent cytokine-induced insulin secretion failure in human pancreatic islets. Human islets were exposed to culture medium containing 5.5 mM glucose in the presence or in the absence of cytokines. They observed that the insulin secretion in response to glucose (stimulation index in the graph) was significantly decreased by ˜40-50% in human islets exposed for 72 h to the mixture of cytokines (FIG. 12). They further investigated whether treatment of human islets with the Tpl2 inhibitor protects them from insulin secretion dysfunction. They observed that treating the human islets with a non cytotoxic concentration of the Tpl2 inhibitor significantly prevented cytokine-induced insulin secretion failure by ˜50% (FIG. 12), indicating that the human islets treated with the Tpl2 inhibitor are more viable and functional, and protected (but partially) against the deleterious effects of an inflammatory cytokine mix.

Importantly, combination of Tpl2 inhibitor and Exendin-4 treatment totally prevented cytokine-induced insulin secretion failure (FIG. 7), indicating that the human islets treated with combination of Tpl2 inhibitor and Exendin-4 are viable and functional, and totally protected against the detrimental effects of cytokines on β-cell glucose sensing and insulin secretion.

### Example 4

### Combination of Tpl2 Kinase Inhibitor and GPL-1 Agonist (Liraglutide) Useful for Preventing or Treating Diabetes

Combination of Tpl2 inhibitor and the GLP-1 analogue, Liraglutide, protects INS-1E cells from cytokine induced apoptosis

Material & Methods

INS-1E β-cells were treated in RPMI medium containing 7.5% SVF with or without Tpl2 inhibitor (3 μM) and/or Liraglutide (20 nM) during 2 h and then stimulated or not with Tpl2 inhibitor (3 μM), Liraglutide (20 nM) and/or a cytokine mix (IL-1β (0.2 ng/ml), TNF-α (50 ng/ml), and IFN-γ (30 ng/ml) for 24 h. Lysates were subjected to Western blotting with antibodies against cleaved caspase-3 (1:1000) or β-actin (1:5000).

Results

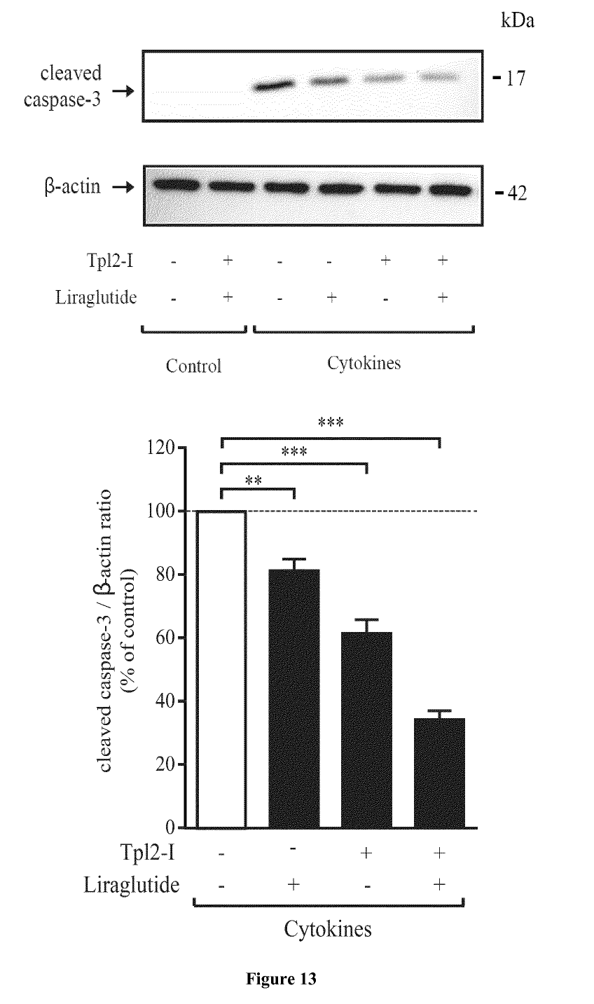

Liraglutide alone or a liraglutide/Tpl2 inhibitor combination were investigated on INS-1E cells submitted to the deleterious effects of cytokines. We found that combination of pharmacological inhibition of Tpl2 and liraglutide produces powerful anti-apoptotic effects on INS-1E cells than each compound alone (Reduction by 70/80% of cleaved caspase-3 induced by the cytokine mix) (FIG. 13).

### Example 5

### Combination of Tpl2 Kinase Inhibitor and DPP-4 Inhibitor (Sitagliptin) Useful for Preventing or Treating Diabetes

Combination of Tpl2 inhibitor and dipeptidyl peptidase-4 inhibitor (DDP-4) (Sitagliptin) protects INS-1E cells from cytokine induced apoptosis.

Material & Methods

INS-1E β-cells were treated in RPMI medium containing 7.5% SVF with or without Tpl2 inhibitor (3 μM) and/or GLP-1 (20 nM), DPP-4 inhibitor (Sitagliptin, 20 nM) during 2 h and then stimulated or not with Tpl2 inhibitor (3 μM), GLP-1 (20 nM), DPP-4 inhibitor (Sitagliptin, 20 nM) and/or a cytokine mix (IL-1β (0.2 ng/ml), TNF-α (50 ng/ml), and IFN-γ (30 ng/ml) for 24 h. Lysates were subjected to Western blotting with antibodies against cleaved caspase-3 (1:1000) or β-actin (1:5000).

Results

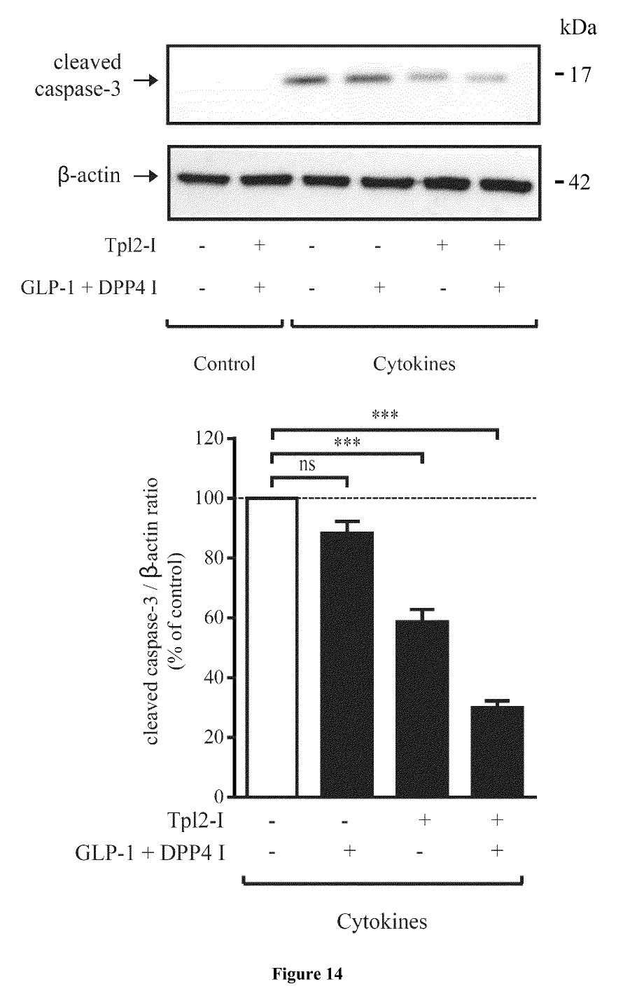

GLP-1 (20 nM) and DPP-4 inhibitor (Sitagliptin, 20 nM) or a GLP-1 (20 nM)/DPP4 inhibitor (Sitagliptin, 20 nM) and Tpl2 inhibitor (3 μM) combination were investigated on INS-1E cells submitted to the deleterious effects of cytokines. We found that combination of pharmacological inhibition of Tpl2 and GLP-1/DPP4 inhibitor produces powerful anti-apoptotic effects on INS-1E cells than each compound alone (Reduction by 80% of cleaved caspase-3 induced by cytokines) (FIG. 14).

Discussion

A major focus of type 2 diabetes research in recent years has been to elucidate the disease pathogenesis. It has become clear that chronic inflammation is a hallmark of T2DM, affecting both β-cell function and mass (Donath et al, 2011). Immunomodulatory strategies for the treatment of T2DM have emerged (Böni-Schnetzler et al, 2012; Larsen et al, 2009; Larsen et al, 2007). Reduced hyperglycaemia and improved β-cell function were observed in T2D patients treated with IL-1β receptor antagonist (IL-1RA) to solely block the deleterious effects of IL-1β (Larsen et al, 2007).

The inventors provide the first evidences that the use of Tpl2 kinase inhibitors may be key therapeutic compounds to alleviate β-cell failure induced not only by IL-1β, but also by a pro-apoptotic cytokine mixture (IL-1β, TNF-α, IFN-γ). Hence, using Tpl2 kinase inhibitors may have the potential to slow or stop the advance of T2DM by reducing β-cell failure and destruction induced by chronic inflammation, thus providing further support to it acts on the pathogenesis of the disease. Consequently, targeting Tpl2 kinase may have the potential to exert a major impact in benefitting patients suffering from T2D by reducing disease symptoms and complications. This, on turn, will help reduce the growing healthcare and social burden caused by the complications of T2DM, such as nephropathy, neuropathy, eye damage or cardiovascular disease.

Pre-clinical and clinical researches demonstrated that GLP-1 receptor agonists (such as Exendin-4) have modest anti-inflammatory effects in addition to that of improving β-cell survival. They discovered that the protective effect of Exendin-4 against inflammatory cytokines action in human islets is enhanced by Tpl2 kinase inhibitor. Indeed, the combined use of Exendin-4 and Tpl2 kinase inhibitor compounds enhances β-cell and human pancreatic islet viability and function in the presence of inflammatory cytokines and could be used as more powerful and more pleiotropic anti-diabetic treatment. Based on the present results, combination of GLP-1 receptor agonist and Tpl2 kinase inhibitor may represent a novel therapeutic strategy and benefits for the treatment of T2DM. This novel pharmacological approach may act on the pathogenesis of the disease rather than just on its symptoms.

Using Tpl2 kinase inhibitors may also represent a potential therapeutic benefit in pancreatic islet transplantation procedure. Indeed, 80% transplanted islets die during the post-transplantation period by apoptosis due to IBMIR mediated especially by a mixture of cytokines including IL-1β, TNF-α and IFN-γ (Nilsson et al, 2011; van der Windt et al, 2007). Hence, the importance of blocking IBMIR in terms of islet engraftment and increased success rates in islet transplantation is currently highlighted (Nilsson et al, 2011; van der Windt et al, 2007). Targeting Tpl2 kinase may represent a strategy that is clinically applicable to prevent IBMIR.

Collectively, the present results not only propose Tpl2 kinase inhibitors as therapeutic compounds to alleviate β-cell failure observed in T2DM, but also provide important new insights into the molecular mechanisms that promote β-cell dysfunction, the damaging effects of inflammation in β-cells. These results favour the exhaustive analysis of the signalling molecular mechanisms specifically engaged by pro-inflammatory cytokines permitting the identification of novel anti-diabetic targets, which may be amenable for further drug development. Finally, these results reinforce the industrial development of new Tpl2 kinase inhibitors with great efficacy in vivo which will permit their development as novel anti-diabetic drugs.

### REFERENCES

Throughout this application, various references describe the state of the art to which this invention pertains. The disclosures of these references are hereby incorporated by reference into the present disclosure.
Aoki M, Hamada F, Sugimoto T, Sumida S, Akiyama T, Toyoshima K. The human cot proto-oncogene encodes two protein serine/threonine kinases with different transforming activities by alternative initiation of translation. J Biol Chem. 1993 Oct. 25; 268(30):22723-32.
Böni-Schnetzler M, Donath M Y. How biologics targeting the IL-1 system are being considered for the treatment of type 2 diabetes. Br J Clin Pharmacol. 2012 Apr. 17. doi: 10.1111/j.1365-2125.2012.04297.
Bucher P, Mathe Z, Morel P, Bosco D, Andres A, Kurfuest M, Friedrich O, Raemsch-Guenther N, Buhler L H, Berney T. Assessment of a novel two-component enzyme preparation for human islet isolation and transplantation. Transplantation. 2005 Jan. 15; 79(1):91-7.
Burke J P, Williams K, Gaskill S P, Hazuda H P, Haffner S M, Stern M P. Arch Int Med 159: 1450-1456, 1999.
Butler A E, Janson J, Bonner-Weir S, Titzel R, Rizza R A, Butler P C. Diabetes 52: 102-110, 2003.
Center for Disease Control and Prevention: Trends in the prevalence and incidence of self reported diabetes mellitus: United States, 1980-1994. MMWR 46: 1014-1018, 1997.
Costes S, Broca C, Bertrand G, Lajoix A D, Bataille D, Bockaert J, Dalle S. ERK1/2 control phosphorylation and protein level of cAMP-responsive element-binding protein: a key role in glucose-mediated pancreatic β-cell survival. Diabetes. 2006 August; 55(8):2220-30.
DeFronzo R A. Diabetes 37: 667-687, 1988
Dinarello C A, Donath M Y, Mandrup-Poulsen T. Role of IL-1β in type 2 diabetes. Curr Opin Endocrinol Diabetes Obes. 2010 August; 17(4):314-21. Review.
Donath M Y, Shoelson S E. Type 2 diabetes as an inflammatory disease. Nat Rev Immunol. 2011 February; 11(2):98-107.
Ehses J A, Lacraz G, Giroix M H, Schmidlin F, Coulaud J, Kassis N, Irminger J C, Kergoat M, Portha B, Homo-Delarche F, Donath M Y. IL-1 antagonism reduces hyperglycemia and tissue inflammation in the type 2 diabetic GK rat. Proc Natl Acad Sci USA. 2009 Aug. 18; 106(33):13998-4003.
Gantke T, Sriskantharajah S, Ley S C. Regulation and function of TPL-2, an IκB kinase-regulated MAP kinase kinase kinase. Cell Res. 2011 January; 21(1):131-45.
George D, Salmeron A. Cot/Tp1-2 protein kinase as a target for the treatment of inflammatory disease. Curr Top Med Chem. 2009; 9(7):611-22.
Kaila N, Green N, Li H Q, Hu Y, Janz K, Gavrin L K, Thomason J, Tam S, Powell D, Cuozzo J, Hall J P, Telliez J B, Hsu S, Nickerson-Nutter C, Wang Q, Lin L L. Identification of a novel class of selective Tpl2 kinase inhibitors: 4-Alkylamino-[1,7]naphthyridine-3-carbonitriles. Bioorg Med Chem. 2007 Oct. 1; 15(19):6425-42.
Kudva Y C, Butler P C. In Clinical Research in Diabetes and Obesity. Vol. 2: Diabetes and Obesity. Draznin B, Rizza R, Eds. Totowa, N.J., Humana Press, 1997, p. 119-136.
Lancaster G I, Kowalski G M, Estevez E, Kraakman M J, Grigoriadis G, et al. (2012) Tumor Progression Locus 2 (Tpl2) Deficiency Does Not Protect against Obesity-Induced Metabolic Disease. PLoS ONE 7(6): e39100. doi:10.1371/journal.pone.0039100
Larsen C M, Faulenbach M, Vaag A, Ehses J A, Donath M Y, Mandrup-Poulsen T. Sustained effects of interleukin-1 receptor antagonist treatment in type 2 diabetes. Diabetes Care. 2009 September; 32(9):1663-8. Epub 2009 Jun. 19.
Larsen C M, Faulenbach M, Vaag A, Volund A, Ehses J A, Seifert B, Mandrup-Poulsen T, Donath M Y. Interleukin-1-receptor antagonist in type 2 diabetes mellitus. N Engl J Med. 2007 Apr. 12; 356(15):1517-26.
Leibowitz G, Kaiser N, and Cerasi E, Diabetes, Obesity and Metabolism, 11 (Suppl. 4), 1-9, 2009.
Ludvik B, Nolan J J, Baloga J, Sacks D, Olefsky J. Diabetes 44: 1121-1125, 1995.
Maedler K, Storling J, Sturis J, Zuellig R A, Spinas G A, Arkhammar P O, Mandrup-Poulsen T, Donath M Y. Glucose- and interleukin-1β-induced β-cell apoptosis requires Ca2+ influx and extracellular signal-regulated kinase (ERK) 1/2 activation and is prevented by a sulfonylurea receptor 1/inwardly rectifying K+ channel 6.2 (SUR/Kir6.2) selective potassium channel opener in human islets. Diabetes. 2004 July; 53(7):1706-13.
Maedler K, Sergeev P, Ris F, Oberholzer J, Joller-Jemelka H I, Spinas G A, Kaiser N, Halban P A, Donath M Y. Glucose-induced β cell production of IL-1β contributes to glucotoxicity in human pancreatic islets. J Clin Invest. 2002 September; 110(6):851-60.
Nilsson B, Ekdahl K N, Korsgren O. Control of instant blood-mediated inflammatory reaction to improve islets of Langerhans engraftment. Curr Opin Organ Transplant. 2011 December; 16(6):620-6.
Perfield J W 2nd, Lee Y, Shulman G I, Samuel V T, Jurczak M J, Chang E, Xie C, Tsichlis P N, Obin M S, Greenberg A S. Tumor progression locus 2 (TPL2) regulates obesity-associated inflammation and insulin resistance. Diabetes. 2011 April; 60(4):1168-76.
Polonsky K S. Int J Obes Relat Metab Disord 24 (Suppl. 2): S29-S31, 2000.
Portha B, Lacraz G, Kergoat M, Homo-Delarche F, Giroix M H, Bailbé D, Gangnerau M N, Dolz M, Tourrel-Cuzin C, Movassat J. The GK rat β-cell: a prototype for the diseased human β-cell in type 2 diabetes? Mol Cell Endocrinol. 2009 Jan. 15; 297(1-2):73-85.
Pugazhenthi U, Velmurugan K, Tran A, Mahaffey G, Pugazhenthi S. Anti-inflammatory action of exendin-4 in human islets is enhanced by phosphodiesterase inhibitors: potential therapeutic benefits in diabetic patients. Diabetologia. 2010 November; 53(11):2357-68. Epub 2010 Jul. 16.
Ricordi C, Lacy P E, Finke E H, Olack B J, Scharp D W. Automated method for isolation of human pancreatic islets. Diabetes. 1988 April; 37(4):413-20.
van der Windt D J, Bottino R, Casu A, Campanile N, Cooper D K. Rapid loss of intraportally transplanted islets: an overview of pathophysiology and preventive strategies. Xenotransplantation. 2007 July; 14(4):288-97.
Vougioukalaki M, Kanellis D C, Gkouskou K, Eliopoulos A G. Tpl2 kinase signal transduction in inflammation and cancer. Cancer Lett. 2011 May 28; 304(2):80-9.
Wu J, Green N, Hotchandani R, Hu Y, Condon J, Huang A, Kaila N, Li H Q, Guler S, Li W, Tam S Y, Wang Q, Pelker J, Marusic S, Hsu S, Perry Hall J, Telliez J B, Cui J, Lin L L. Selective inhibitors of tumor progression loci-2 (Tpl2) kinase with potent inhibition of TNF-alpha production in human whole blood. Bioorg Med Chem Lett. 2009 Jul. 1; 19(13):3485-8.

1. A Tumor Progression Locus-2 (Tpl2) kinase inhibitor for use in the prevention or treatment of diabetes in a patient in need thereof.

2. An inhibitor of the Tpl2 kinase gene expression for use in the prevention or treatment of diabetes in a patient in need thereof.

3. A Tpl2 kinase inhibitor or an inhibitor of the Tpl2 kinase gene expression for use in improving survival or function of pancreatic P-cells in a patient in need thereof.

4. A Tpl2 kinase inhibitor or an inhibitor of the Tpl2 kinase gene expression for use in the prevention of type 2 diabetes mellitus (TD2M) in a patient in need thereof.

5. The Tpl2 kinase inhibitor or the inhibitor of the Tpl2 kinase gene expression for use according to claim 1, wherein said patient is a lean patient.

6. The Tpl2 kinase inhibitor or the inhibitor of the Tpl2 kinase gene expression for use according to claim 1, wherein said Tpl2 kinase inhibitor is 4-(3-cloro-4-fluorophenylamino)-6-(pyridine-3-yl-methylamino)-3-cyano-[1,7]-napthyridine.

7. A pharmaceutical composition or a kit comprising a Tpl2 kinase inhibitor or an inhibitor of the Tpl2 kinase gene expression and an anti-diabetic drug.

8. The pharmaceutical composition or the kit according to claim 7, wherein the anti-diabetic drug is a glucagon-like peptide-1 (GLP-1) receptor agonist.

9. The pharmaceutical composition or the kit according to claim 7, wherein the anti-diabetic drug is a glucagon-like peptide-1 (GLP-1) receptor agonist and wherein the GLP-1 receptor agonist is exendin-4, exenatide, or liraglutide.

10. The pharmaceutical composition or the kit according to claim 7, wherein the anti-diabetic drug is a dipeptidyl peptidase-4 (DDP-4) inhibitor.

11. The pharmaceutical composition or the kit according to claim 7, wherein the anti-diabetic drug is a dipeptidyl peptidase-4 (DDP-4) inhibitor and wherein the DDP-4 inhibitor is sitagliptin.

12. A Tpl2 kinase inhibitor or an inhibitor of the Tpl2 kinase gene expression for use in enhancing the clinical efficacy of an anti-diabetic drug.

13. A Tpl2 kinase inhibitor or an inhibitor of the Tpl2 kinase gene expression for use in enhancing the anti-inflammatory action and/or the preservation of pancreatic P-cell viability and/or function of an anti-diabetic drug.

14. A culture medium suitable for the culture of mammalian pancreatic |3-cells comprising a Tpl2 kinase inhibitor or an inhibitor of the Tpl2 kinase gene expression.

15. The culture medium according to claim 14, further comprising 5.6 mmol/l glucose, 10% fetal bovine serum (FBS) or human serum albumin (HSA), 100 UI/ml penicillin, 100 mg/ml streptomycin and 2 mM glutamine.

16. A method for improving survival and/or function of a population of pancreatic P-cells in vitro or ex vivo, said method comprising a step of contacting said population with a culture medium comprising an effective amount of a Tpl2 kinase inhibitor or an inhibitor of the Tpl2 kinase gene expression.

17. A method for improving survival and/or function of a pancreatic P-cell transplant, said method comprising a step of administering an effective amount of Tpl2 kinase inhibitor or an inhibitor of the Tpl2 kinase gene expression to a patient with a pancreatic P-cell transplant.

18. A Tpl2 kinase inhibitor or an inhibitor of the Tpl2 kinase gene expression for use in the prevention or treatment of instant blood-mediated inflammatory reaction (IBMIR) in a patient with a pancreatic P-cell transplant.

19. A method for preventing or treating diabetes comprising administering to a patient in need thereof an effective amount of a Tpl2 kinase inhibitor or an inhibitor of the Tpl2 kinase gene expression.

20. A method for improving survival or function of pancreatic β-cells in a patient comprising administering to a patient in need thereof an effective amount of a Tpl2 kinase inhibitor or an inhibitor of the Tpl2 kinase gene expression.

21. The method according to claim 19 or claim 20, wherein said patient is a lean patient.

22. The method according to claim 19, wherein said diabetes is TD2M.

23. The method according to claim 19 or claim 20, wherein said Tpl2 kinase inhibitor is 4-(3-cloro-4-fluorophenylamino)-6-(pyridine-3-yl-methylamino)-3-cyano-[1,7]-napthyridine.
